(19) United States
(12) Patent Application Publication
(10) Pub. No.: US 2017/0216166 A1
(43) Pub. Date: Aug. 3, 2017

(54) CAPSULES CONTAINING POLYVINYL ALCOHOL

(71) Applicant: International Flavors & Fragrances Inc., New York, NY (US)

(72) Inventors: Takashi SASAKI, Matawan, NJ (US); Johan Gerwin Lodewijk PLUYTER, Middletown, NJ (US); Crystal KUNZEL, Barnegat, NJ (US); Volkert DE VILLENEUVE, Vooburg (NL); Mikal VAN LEEUWEN, Utrecht (NL); Xantha KNEVEL, Lelystad (NL); Na YUE, Hilversum (NL); Yuchuan LIN, East Brunswick, NJ (US)

(21) Appl. No.: 15/514,875

(22) PCT Filed: Oct. 1, 2015
(86) PCT No.: PCT/US15/53456

§ 371 Date: Mar. 28, 2017

Related U.S. Application Data
(60) Provisional application No. 62/058,388, filed on Oct. 1, 2014.

(51) Int. Cl. A61K 8/11; A61K 8/81; C11D 3/50; A61Q 5/02; A61Q 19/10; C11D 3/37; A61K 8/73; A61K 8/87

(52) U.S. Cl. CPC A61K 8/11; A61K 8/731; A61K 8/8129; A61K 8/87; A61Q 5/02; A61Q 19/10; C11D 3/3753; C11D 3/505; A61K 2800/412; A61K 2800/56

(57) ABSTRACT

A method of preparing a capsule composition by encapsulating an active material such as a fragrance or flavor in the presence of a dispersant that includes a fully hydrolyzed polyvinyl alcohol and a water-dispersible polymer. Also disclosed are capsule compositions prepared by the method and consumer products containing such a capsule composition.

### CROSS-REFERENCE TO RELATED APPLICATION

This application is a national stage entry under 35 USC 371 for International Application No. PCT/US2015/053456, filed on Oct. 1, 2015, which claims priority to U.S. Application No. 62/058,388, filed on Oct. 1, 2014. The contents of both applications are incorporated herein by reference in their entirety.

### BACKGROUND

Active materials are used in numerous products to enhance the consumer's enjoyment of the products such as laundry detergents, fabric softeners, soaps, personal care products including shampoos, body washes, deodorants and the like, as well as certain food products. These active materials include fragrances, flavors, and malodor counteracting agents.

The delivery effectiveness of the active materials can be enhanced by encapsulating them in nano- or macro-capsules so that these materials are released at a desired time, e.g., at damp when a consumer open the door of a washing machine right after washing laundry. In the capsules, polymeric walls protect the active material from evaporation, reaction, oxidation or otherwise dissipating prior to use. For example, U.S. Pat. No. 4,081,384 discloses a softener or anti-stat core coated by a polycondensate suitable for use in a fabric conditioner. U.S. Pat. No. 5,112,688 discloses selected fragrance materials having the proper volatility to be coated by coacervation with microparticles in a wall that can be activated for use in fabric conditioning. U.S. Pat. No. 5,145,842 discloses a solid core of a fatty alcohol, ester, or other solid plus a fragrance coated by an aminoplast shell. U.S. Pat. No. 6,248,703 discloses various agents including fragrance in an aminoplast shell that is included in an extruded bar soap.

However, current capsule compositions do not provide optimized release profiles in all applications such as an intensive laundry washing cycle in a European washing machine.

In addition, known methods of preparing capsules can seldom control the capsule particle size, an important factor determining the release profile and stability of capsules. Suitable capsules for fragrance deliver include those having a desired particle size range. See US Patent Application 20080311064. A large capsule may not be able to maintain its physical integrity, making it unstable. On the other hand, a small capsule can be hard to break or release a fragrance. To tackle this problem, U.S. Pat. No. 6,890,592 teaches a process of preparing microcapsules having a substantially uniform size distribution by extruding a fragrance core oil through a membrane under high pressure. This process is not suitable for all capsule wall materials and also consumes a significant amount of energy.

There is a need to develop a method for preparing capsule composition with optimized organoleptic/release profiles and particle sizes suitable for a wide variety of consumer applications.

### SUMMARY OF THE INVENTION

This invention is based on an unexpected discovery that certain capsules containing a fully hydrolyzed polyvinyl alcohol possess desirable properties including high perceived olfactory intensity.

Accordingly, one aspect of this invention relates to a capsule containing: (i) an oil core having an active material (e.g., a fragrance, flavor, malodor counteracting agent, and combination thereof); (ii) an capsule wall formed of a wall-forming material, the capsule wall encapsulates the oil core; and (iii) a dispersant containing a fully hydrolyzed polyvinyl alcohol and a water-dispersible polymer, both of which are immobilized in the capsule wall.

The capsules each have a particle size in the range of 0.1 to 1000 microns (e.g., 0.2 to 500 microns, 0.3 to 300 microns, 0.5 to 200 microns, 5 to 150 microns, 10 to 100 microns, and 20 to 70 microns).

The weight ratio between the fully hydrolyzed polyvinyl alcohol and the water-dispersible polymer is preferably 1:10 to 100:1 (e.g., 1:5 to 10:1 and 3:10 to 5:1).

The fully hydrolyzed polyvinyl alcohol typically has a degree of hydrolysis of 96% or greater, and preferably, 98% or greater.

The water-dispersible polymer can be dispersed in water to form a colloidal suspension or a polymeric solution. Examples include a polysaccharide, protein, polypeptide, polyacrylate, polyolefin, polyurethane, polyurea, polyamide, polyalkylene oxide, polysiloxane, polyamine, and combination thereof. Suitable polysaccharide can be agar, carboxymethylcellulose, carboxyethylcellulose, alginic acid, xyloglucan, xanthum gum, gum Arabic, hydroxypropyl cellulose, hydroxyethyl cellulose, carrageenan, modified starch, modified cellulose, galactomannans, amphoteric guar, hydrophobically modified cationic guar, hydrophobically modified amphoteric guar, hydrophobically modified anionic guar, bacterial alginate, fucogalactan, fucoidan, gellan gum, gum ghatti, gum karaya, gum tragacanth, pectin, propylene glycol alginate, psyllium seed gum, sodium alginate, welan gum, and any combination thereof.

The capsule wall is typically formed of a polyacrylate, polyurea, polyurethane, polyacrylamide, polyester, polyether, polyamide, poly(acrylate-co-acrylamide), starch, silica, gelatin and gum Arabic, poly(melamine-formaldehyde), poly(urea-formaldehyde), or combination thereof. Preferably, it is a polyurea, polyurethane, or combination thereof.

The capsules can have a wall including an outer layer and an inner layer.

Another aspect of this invention relates to a method of preparing a capsule composition containing a fully hydrolyzed polyvinyl alcohol. The method includes the steps of: (a) providing an oil phase containing an active material and a wall-forming material; (b) providing an aqueous phase containing a dispersant that includes a fully hydrolyzed polyvinyl alcohol and a water-dispersible polymer; (c) emulsifying the oil phase into the aqueous phase to form an oil-in-water emulsion; (d) optionally adding an activation agent to the oil-in-water emulsion; (e) causing the formation of capsules having an oil core that contains the active material and a capsule wall that is formed of the wall-forming material; and (0 curing the capsules to obtain the capsule composition (e.g., at a temperature of 15° C. to 130° C., 15° C. to 45° C., 45° C. to 130° C., 55° C. to 90° C., or 90-130° C.).

The particle size of the capsules is adjustable by varying the weight ratio between the fully hydrolyzed polyvinyl alcohol and the water-dispersible polymer, which can be 1:10 to 100:1 (e.g., 1:5 to 10:1 and 3:10 to 5:1).

The fully hydrolyzed polyvinyl alcohol, the water-dispersible polymer, and the capsule walls are described above. When the capsule wall is a polyurea, it can be formed by an interfacial polymerization between a polyisocyanate and an amine cross-linker that contains two or more amine groups. The polyurethane capsule wall can be formed by an interfacial polymerization between a polyisocyanate and an alcohol cross-linker that contains two or more hydroxyl groups. Both the polyurea and polyurethane capsule walls can also be formed by an interfacial polymerization between a polyisocyanate and a hybrid cross-linker that contains one or more amine groups and one or more hydroxyl groups.

Also within the scope of this invention is a capsule composition prepared by the method described above.

Still within the scope of this invention is a consumer product containing a capsule composition of this invention and optionally containing one or more different capsules, a free active material, a deposition aid, or a combination thereof. Examples of the consumer products include a shampoo, hair conditioner, personal wash (body wash), liquid fabric detergent, solid fabric detergent, softener, scent booster, bar soap, or hard surface cleaners. When the consumer product is a fabric detergent, it further contains a fabric detergent active.

The details of one or more embodiments of the invention are set forth in the description and drawings below. Other features, objects, and advantages of the invention will be apparent from the description, drawings, and claims.

### BRIEF DESCRIPTION OF THE DRAWINGS

FIG. 1 shows the microscope image of capsules in Composition 1 of this invention.

FIG. 2 shows the Scanning Electron Microscopy (SEM) image of capsules in Composition 1 of this invention.

### DETAILED DESCRIPTION OF THE INVENTION

As described above, the capsule of this invention is prepared using a fully hydrolyzed polyvinyl alcohol and a water-dispersible polymer as a dispersant.

A polyvinyl alcohol is typically prepared in two steps: (i) polymerizing vinyl acetate to obtain a polyvinylacetate, and (ii) hydrolyzing the polyvinylacetate into a polyvinyl alcohol. The degree of hydrolysis of the polyvinyl alcohol is expressed as percentage of hydrolysis of polyvinyl acetate. A fully hydrolyzed polyvinyl alcohol refers to 95% or greater (preferably, 96% or greater, and more preferably, 98% or greater) of the acetate group in the corresponding polyvinylacetate is hydrolyzed. It typically has a molecular weight of 1,000 to 500,000 Dalton (e.g., 2,000 to 250,000 Dalton and 5,000 to 200,000 Dalton). As used in this invention, the fully hydrolyzed polyvinyl alcohol is used and present in the capsule at a level of 0.01% to 25%, more preferably 0.1% to 20% by the weight of the capsule composition containing the capsule.

A fully hydrolyzed polyvinyl alcohol by itself is not an ideal dispersant. As shown in the examples below, capsules could not be prepared using only a fully hydrolyzed polyvinyl alcohol as the dispersant. Indeed, known methods of preparing capsules employ partially hydrolyzed polyvinyl alcohols (e.g., 83% hydrolyzed polyvinyl alcohol such as Mowiol 3-83). See US Patent Application Publications 2013/0330292 and 2013/0337023.

It is unexpectedly found that a fully hydrolyzed polyvinyl alcohol, when combined with a water-dispersible polymer, can be used to prepare capsules having improved fragrance delivery performance in fabric detergents.

Examples of water-dispersible polymers include polysaccharide-based polymers such as carboxymethylcelluloses and their salts, alginic acids and their salts, xyloglucan, xanthum gum, gum arabic, hydroxypropyl cellulose, hydroxyethyl cellulose, carboxymethylcelluloses and their salts, carrageenan, modified starch and celluloses (anionic, cationic and/or non-ionic), galactomannans oxidized starch and cellulose, and dialdehyde starch and cellulose, amphoteric guar, hydrophobically modified cationic guar, hydrophobically modified amphoteric guar, hydrophobically modified anionic guar, such as cationic starch, Celquats (from AKZO-Nobel), Jaguars (from Rhodia), Sensomer CI-50 (from Lubrizol), Softcat (from DOW), Poly SugaQuat (from Colonial Chemical Corp.), Chargemaster starches (from GPC), Hi-Cat starches (from Roquette). Other suitable water-dispersible polymers are natural based polymers such as proteins and hydrolyzed proteins with the pH of the medium adjusted such that their overall charge is negative or positive. Synthetic water soluble polymers can also be used, such as acrylates, olefins, polyurethanes, polyureas, polyamides, polyalkylene oxides, and polysiloxanes, polyamines, and mixtures thereof. These polymers can contain anionic repetitive units formed from acrylic acid, methacrylic acid, maleic anhydride, ethylene maleic anhydride, sulfonated monomers such as styrene sulfonate, sulphated monomers and phenolic monomers, or phosphonate containing monomers. They can also contain cationic repetitive units formed from diallyldimethylammonium chloride, methacrylamidopropyltrimethylammonium chloride, N,N-dimethyl amino ethyl methacrylate, vinyl pyridine, quaternized vinyl pyridine, vinyl amine, allyl amine, vinyl imidazoline, vinyl imidazole, vinyl imidazolinium, dimethylaminoethyl methacrylate, dimethylaminopropyl, or methacryloylaminopropyl lauryldimonium chloride, and amine-based monomers. Polymers can also contain both anionic and cationic repetitive units and thus be amphoteric in nature. A skilled person in the art would be able to readily determine the ratio between the anionic and cationic repetitive units and the pH value at which the polymer is positively or negatively charged.

When CMC is used as a water-dispersible polymer, it has a molecular weight range between 90,000 and 2,500,000 Daltons (e.g., between 90,000 and 1,500,000 Daltons, between 250,000 and 2,500,000, 500,000 and 2,000,000, between 250,000 and 750,000 Daltons, and between 400,000 and 750,000 Daltons). The carboxymethyl cellulose polymer has a degree of substitution between 0.1 and 3, preferably between 0.65 and 1.4, and more preferably between 0.8 and 1. The carboxymethyl cellulose polymer is present typically in the capsule at a level of 0.01% to 25% (preferably 0.01% to 10%, and more preferably 0.01% to 5%) by the weight of the capsule composition.

Any of the above polymers can have a backbone of a random copolymer, graft, block, multi-block, or star architecture. They can be used a fully hydrolyzed polyvinyl alcohol as a dispersant either separately or in any combination.

Capsules can be prepared following known procedures using the dispersant described above. See US 2013/0330292, US 2014/0017287 and WO 2003/101606A1. The newly formed capsules can then be cured at a temperature in the range of, e.g., 15° C. to 160° C. (such as 55° C. to 130° C., 55° C. to 90° C., 55° C. to 75° C., and 90° C. to 130° C.) for 1 minute to 10 hours (e.g., 0.1 hours to 5 hours, 0.2 hours to 4 hours and 0.5 hours to 3 hours). A skilled person in the art can determine, without undue experiments, the curing temperature, duration, and the heating rate.

Not to be bound by any theory, it is believed that there is a direct relationship between higher cure temperature and less leaching of active material from the capsule. Accordingly, the capsules can be cured at a temperature at or above 100° C. (e.g., above 110° C. and 120° C.) to improve the retention capabilities of the capsules.

To obtain capsules with more leaching of the active material, the capsules are cured at or less than 100° C. (e.g., at or less than 90° C. and at or less than 80° C.).

The rate at which the capsules are heated, i.e., cured, also affect the fragrance release profile of the capsules. For instance, the capsules are heated to a target cure temperature at a linear rate of 0.5 to 2° C. per minute (e.g., 1 to 5° C. per minute, 2 to 8° C. per minute, and 2 to 10° C. per minute) over a period of 1 to 60 minutes (e.g., 1 to 30 minutes). The following heating methods may be used: conduction for example via oil, steam radiation via infrared, and microwave, convection via heated air, steam injection and other methods known by those skilled in the art. The target cure temperature used herein refers to the minimum temperature in degrees Celsius at which the capsules may be cured to retard leaching.

The above described method can further comprise the step of (g): adding a salt of a multivalent cation after step (0 to form aggregates each containing two or more capsules, wherein the water-dispersible polymer contains alginic acid, an alginate salt, or a poly(methyl vinyl ether-co-maleic acid). Examples of the salt including any salts of calcium, magnesium, aluminum, iron, manganese, zinc, cobalt, copper, nickel, titanium, chromium, vanadium, and gold. The weight ratio between the salt and the water-dispersible polymer is 1:50,000 to 50,000:1, preferable 1:5000 to 5000:1, and more preferably 1:1000 to 1000:1.

The above method can further include the step of (h): spray drying the capsules to make a capsule composition in a solid form.

The capsules can also be prepared using microfluidics or a membrane system. See Dendukuri, Advanced Materials 2009, 21, 1-16; and U.S. Pat. Nos. 6,890,592 & 7,122,503.

The capsules thus obtained each can have a wall containing two layers: an outer layer and an inner layer. They typically have a particle size of 0.1 to 1000 microns (e.g., 0.2 to 500 microns, 0.3 to 300 microns, 0.5 to 200 microns, 10 to 100 microns, and 20 to 70 microns). The capsule distribution can be narrow, broad, or multi-modal.

Unexpectedly, one can prepare capsules having a predetermined particle size by varying the weight ratio between the fully hydrolyzed polyvinyl alcohol and the water-dispersible polymer. In general, using a high ratio leads to a smaller particle size as shown in the examples below. A skilled person in the art can choose such as ratio, along with the concentrations of the two polymers and those of the wall-forming material and the core oil, to prepare capsules having a desirable particle size and an improved fragrance release profile. The particle size herein refers to the diameter of a capsule, which is typically spherical.

The capsules can be selected from the following: (i) capsules having a capsule wall formed of urea-formaldehyde, melamine-formaldehyde phenolic-formaldehyde, urea-glutaraldehyde, melamine-glutaraldehyde, phenolic-glutaraldehyde, and any combination thereof; (ii) capsules having a capsule wall formed of polyurea (isocyanate-based), polyurethane, and any combination thereof; (iii) acrylate-based hydrogel core-shell capsules, polyurea/polyurethane-acrylic hybrid core-shell capsules, and any combination thereof; (iv) polyamide-based and/or polyester-based capsules; (v) capsules produced using epoxy-crosslinkers; (vi) capsules based on silica and silica-derived materials which are typically produced using sol-gel processes. Some of these capsules are described in greater detail below.

The capsule compositions each can contain, in addition to the capsules described above, multiple different capsules/polymeric particles, e.g., capsules containing different fragrances, and/or capsules having different wall materials, different particle sizes, or different wall thickness or density for a target application. Suitable capsules are described in detail below.

### Core-Shell Encapsulation Systems

The capsules can be prepared following encapsulation procedures known in the art, see for example U.S. Pat. Nos. 2,800,457, 3,870,542, 3,516,941, 3,415,758, 3,041,288, 5,112,688, 6,329,057, and 6,261,483. Wall forming materials include a melamine formaldehyde, polyurethane, polysiloxanes, polyurea, polyamide, polyimide, polyvinyl alcohol, polyanhydride, polyolefin, polysulfone, polysaccharide, protein, polypeptide, polylactide (PLA), polyglycolide (PGA), polyorthoester, polyphosphazene, silicone, lipid, modified cellulose, gum, polystyrene, polyester, polyether, and combination of these materials. Other polymeric materials that are functional are ethylene maleic anhydride copolymer, styrene maleic anhydride copolymer, ethylene vinyl acetate copolymer, and lactide glycolide copolymer. Biopolymers that are derived from alginate, chitosan, collagen, dextran, gelatin, and starch can also be used as the encapsulating materials. Additionally, capsules can be made via the simple or complex coacervation of gelatin. Preferred encapsulating wall polymers include those formed from isocyanates, acrylates, acrylamide, acrylate-co-acrylamide, hydrogel monomers, sol-gel precursors, gelatin, melamine-formaldehyde or urea-formaldehyde condensates, as well as similar types of aminoplasts.

### Polyurea/Polyurethane Capsules

Polyurea capsules can be prepared using multi-functional isocyanates and multi-functional amines. See WO 2004/054362; EP 0 148149; EP 0 017 409 B1; U.S. Pat. Nos. 4,417,916, 4,124,526, 4,285,720, 4,681,806, 5,583,090, 6,340,653 6,566,306, 6,730,635, 8,299,011, WO 90/08468, and WO 92/13450.

These isocyanates contain two or more isocyanate (—NCO) groups. Suitable isocyanates include, for example, 1,5-naphthylene diisocyanate, 4,4′-diphenylmethane diisocyanate (MDI), hydrogenated MDI (H12MDI), xylylene diisocyanate (XDI), tetramethylxylol diisocyanate (TMXDI), 4,4′-diphenyldimethylmethane diisocyanate, di- and tetraalkyldiphenylmethane diisocyanate, 4,4′-dibenzyl diisocyanate, 1,3-phenylene diisocyanate, 1,4-phenylene diisocyanate, the isomers of tolylene diisocyanate (TDI), optionally in a mixture, 1-methyl-2,4-diisocyanatocyclohexane, 1,6-diisocyanato-2,2,4-trimethylhexane, 1,6-diisocyanato-2,4,4-trimethylhexane, 1-isocyanatomethyl-3-isocyanato-1,5,5-trimethylcyclohexane, chlorinated and brominated diisocyanates, phosphorus-containing diisocyanates, 4,4′-diisocyanatophenylperfluoroethane, tetramethoxybutane 1,4-diisocyanate, butane 1,4-diisocyanate, hexane 1,6-diisocyanate (HDI), dicyclohexylmethane diisocyanate, cyclohexane 1,4-diisocyanate, ethylene diisocyanate, phthalic acid bisisocyanatoethyl ester, also polyisocyanates with reactive halogen atoms, such as 1-chloromethylphenyl 2,4-diisocyanate, 1-bromomethylphenyl 2,6-diisocyanate, and 3,3-bischloromethyl ether 4,4′-diphenyldiisocyanate. Sulfur-containing polyisocyanates are obtained, for example, by reacting hexamethylene diisocyanate with thiodiglycol or dihydroxydihexyl sulfide. Further suitable diisocyanates are trimethylhexamethylene diisocyanate, 1,4-diisocyanatobutane, 1,2-diisocyanatododecane and dimer fatty acid diisocyanate.

The multi-functional amines contains two or more amine groups including —NH₂ and —RNH, R being substituted and unsubstituted C₁-C₂₀ alkyl, C₁-C₂₀ heteroalkyl, C₁-C₂₀ cycloalkyl, 3- to 8-membered heterocycloalkyl, aryl, and heteroaryl.

Water soluble diamines are one class of amines useful to prepare polyurea capsules as the amines are usually present in the aqueous phase. One class of such amine is of the type:

H₂N(CH₂)ₙNH₂,

where n is ≧1. When n is 1, the amine is a diamine, ethylene diamine. When n is 2, the amine is diamine propane and so on. Exemplary amines of this type include, but are not limited to, ethylenediamine, 1,3-diaminopropane, 1,4-diaminobutane, hexanethylene diamine, hexamethylene diamine, and pentaethylenehexamine. In particular embodiments of this invention, the preferred n is 6, where the amine is a hexamethylene diamine.

Amines that have a functionality greater than 2, but less than 3 and which may provide a degree of cross linking in the shell wall are the polyalykylene polyamines of the type:

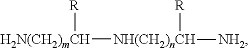

where R equals hydrogen or —CH₃, m is 1-5 and n is 1-5, e.g., diethylene triamine, triethylene tetraamine and the like. Exemplary amines of this type include, but are not limited to diethylenetriamine, bis(3-aminopropyl)amine, bis(hexamethylene)triamine.

Another class of amine that can be used is polyetheramines. They contain primary amino groups attached to the end of a polyether backbone. The polyether backbone is normally based on either propylene oxide (PO), ethylene oxide (EO), or mixed PO/EO. The ether amine can be monoamine, diamine, or triamine, based on this core structure. An example is:

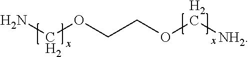

Other exemplary polyetheramines include 2,2′-ethylenedioxy)bis(ethylamine) and 4,7,10-trioxa-1,13-tridecanediamine.

Other suitable amines include tris(2-aminoethyl)amine, triethylenetetramine, N,N′-bis(3-aminopropyl)-1,3-propanediamine, tetraethylene pentamine, 1,2-diaminopropane, N,N,N′,N′-tetrakis(2-hydroxyethyl)ethylene diamine, N,N,N′,N′-tetrakis(2-hydroxypropyl)ethylene diamine, branched polyethylenimine, 2,4-diamino-6-hydroxy-pyrimidine and 2,4,6-triaminopyrimidine.

Amphoteric amines, i.e., amines that can react as an acid as well as a base, are another class of amines of use in this invention. Examples of amphoteric amines include proteins and amino acids such as gelatin, L-lysine, L-arginine, L-lysine monohydrochloride, arginine monohydrochloride and ornithine monohydrochloride.

Guanidine amines and guanidine salts are yet another class of amines of use in this invention. Exemplary guanidine amines and guanidine salts include, but are not limited to, 1,3-diaminoguanidine monohydrochloride, 1,1-dimethylbiguanide hydrochloride, guanidine carbonate and guanidine hydrochloride.

Commercially available examples of amines include JEFFAMINE EDR-148 (where x=2), JEFFAMINE EDR-176 (where x=3) (from Huntsman). Other polyether amines include the JEFFAMINE ED Series, and JEFFAMINE TRIAMINES.

Alcohols of use as cross-linking agents typically have at least two nucleophilic centers. Exemplary alcohols include, but are not limited to, ethylene glycol, hexylene glycol, pentaerythritol, glucose, sorbitol, and 2-aminoethanol.

The preparation of polyurethane capsules can be carried out by reacting one or more of the above-referenced isocyanates with a diol or polyol in the presence of a catalyst. Diols or polyols of use in the present invention have a molecular weight in the range of 200 to 2000 Daltons. Exemplary diols include, but are not limited to, ethylene glycol, diethylene glycol, propylene glycol, 1,4-butane diol, 1,4 hexane diol, dipropylene glycol, cyclohexyl 1,4 dimethanol, and 1,8 octane diol. Exemplary polyols include, but are not limited to, poly (ethylene glycols), poly (propylene glycols), and poly (tetramethylene glycols).

Suitable catalysts include amino or organometallic compounds and include, for example, 1,4-diazabicyclo[2.2.2]octane (e.g., DABCO, Air Products, Allentown, Pa.), N,N-dimethylaminoethanol, N,N-dimethylcyclohexylamine, bis-(2-dimethylaminoethyl) ether, N,N dimethylacetylamine, stannous octoate and dibutyltin dilaurate.

### Aminoplasts and Gelatin

A representative process used for aminoplast encapsulation is disclosed in U.S. Pat. No. 3,516,941, though it is recognized that many variations with regard to materials and process steps are possible. Another encapsulation process, i.e., gelatin encapsulation, is disclosed in U.S. Pat. No. 2,800,457. Both processes are discussed in the context of fragrance encapsulation for use in consumer products in U.S. Pat. Nos. 4,145,184 and 5,112,688 respectively. Polymer systems are well-known in the art and non-limiting examples of these include aminoplast capsules and encapsulated particles as disclosed in Application GB 2006709 A; the production of micro-capsules having walls comprising styrene-maleic anhydride reacted with melamine-formaldehyde precondensates as disclosed in U.S. Pat. No. 4,396,670; an acrylic acid-acrylamide copolymer, cross-linked with a melamine-formaldehyde resin as disclosed in U.S. Pat. No. 5,089,339; capsules composed of cationic melamine-formaldehyde condensates as disclosed in U.S. Pat. No. 5,401,577; melamine formaldehyde microencapsulation as disclosed in U.S. Pat. No. 3,074,845; amido-aldehyde resin in-situ polymerized capsules (see EP 0 158 449 A1); etherified urea-formaldehyde polymers (see U.S. Pat. No. 5,204,185); melamine-formaldehyde microcapsules as described in U.S. Pat. No. 4,525,520; cross-linked oil-soluble melamine-formaldehyde precondensates as described in U.S. Pat. No. 5,011,634; capsule wall material formed from a complex of cationic and anionic melamine-formaldehyde precondensates that are then cross-linked as disclosed in U.S. Pat. No. 5,013,473; polymeric shells made from addition polymers such as condensation polymers, phenolic aldehydes, urea aldehydes or acrylic polymers as disclosed in U.S. Pat. No. 3,516,941; urea-formaldehyde capsules as disclosed in EP 0 443 428 A2; melamine-formaldehyde chemistry as disclosed in GB 2 062 570 A; and capsules composed of polymer or copolymer of styrene sulfonic acid in acid of salt form, and capsules cross-linked with melamine-formaldehyde as disclosed in U.S. Pat. No. 4,001,140.

### Urea-Formaldehyde and Melamine Formaldehyde Capsules

Urea-formaldehyde and melamine-formaldehyde pre-condensate microcapsule shell wall precursors are prepared by means of reacting urea or melamine with formaldehyde where the mole ratio of melamine or urea to formaldehyde is in the range of from about 10:1 to about 1:6, preferably from about 1:2 to about 1:5. For purposes of practicing this invention, the resulting material has a molecular weight in the range of from 156 to 3000. The resulting material may be used ‘as-is’ as a cross-linking agent for the aforementioned substituted or un-substituted acrylic acid polymer or copolymer or it may be further reacted with a C₁-C₆ alkanol, e.g., methanol, ethanol, 2-propanol, 3-propanol, 1-butanol, 1-pentanol or 1-hexanol, thereby forming a partial ether where the mole ratio of melamine/urea:formaldehyde:alkanol is in the range of 1:(0.1-6):(0.1-6). The resulting ether moiety-containing product may be used ‘as-is’ as a cross-linking agent for the aforementioned substituted or un-substituted acrylic acid polymer or copolymer, or it may be self-condensed to form dimers, trimers and/or tetramers which may also be used as cross-linking agents for the aforementioned substituted or un-substituted acrylic acid polymers or co-polymers. Methods for formation of such melamine-formaldehyde and urea-formaldehyde pre-condensates are set forth in U.S. Pat. Nos. 3,516,846 and 6,261,483, and Lee et al. (2002) J. Microencapsulation 19, 559-569.

Examples of urea-formaldehyde pre-condensates useful in the practice of this invention are URAC 180 and URAC 186, trademarks of Cytec Technology Corp. of Wilmington, Del. Examples of melamine-formaldehyde pre-condensates useful in the practice if this invention, include, but are not limited to, CYMEL U-60, CYMEL U-64 and CYMEL U-65, trademarks of Cytec Technology Corp. of Wilmington, Del. It is preferable to use, as the precondensate for cross-linking, the substituted or un-substituted acrylic acid polymer or co-polymer. In practicing this invention, the range of mole ratios of urea-formaldehyde precondensate/melamine-formaldehyde pre-condensate to substituted/un-substituted acrylic acid polymer/co-polymer is in the range of from about 9:1 to about 1:9, preferably from about 5:1 to about 1:5 and most preferably from about 2:1 to about 1:2.

In one embodiment of the invention, microcapsules with polymer(s) composed of primary and/or secondary amine reactive groups or mixtures thereof and cross-linkers can also be used. See US 2006/0248665. The amine polymers can possess primary and/or secondary amine functionalities and can be of either natural or synthetic origin. Amine-containing polymers of natural origin are typically proteins such as gelatin and albumen, as well as some polysaccharides. Synthetic amine polymers include various degrees of hydrolyzed polyvinyl formamides, polyvinylamines, polyallyl amines and other synthetic polymers with primary and secondary amine pendants. Examples of suitable amine polymers are the LUPAMIN series of polyvinyl formamides available from BASF. The molecular weights of these materials can range from 10,000 to 1,000,000 Daltons.

Urea-formaldehyde or melamine-formaldehyde capsules can also include formaldehyde scavengers, which are capable of binding free formaldehyde. When the capsules are for use in aqueous media, formaldehyde scavengers such as sodium sulfite, melamine, glycine, and carbohydrazine are suitable. When the capsules are aimed to be used in products having low pH, e.g., fabric care conditioners, formaldehyde scavengers are preferably selected from beta diketones, such as beta-ketoesters, or from 1,3-diols, such as propylene glycol. Preferred beta-ketoesters include alkyl-malonates, alkyl aceto acetates and polyvinyl alcohol aceto acetates.

### Capsule Formation Aids

In some embodiments, the fragrance is encapsulated by a polymer in the presence of a capsule formation aid, e.g., a surfactant, emulsifier, or co-dispersant, in addition to the dispersant including the fully hydrolyzed polyvinyl alcohol and water-dispersible polymer. Typically, these capsule formation aids are added to the aqueous phase and assist the emulsifying step.

Most capsule formation aids are used as dispersants (namely, emulsifiers or surfactants). They facilitate the formation of stable emulsions containing nano- or micro-sized oil drops to be encapsulated. Further, capsule formation aids improve the performance of the capsule composition by stabilizing capsules and/or their deposition to the target areas or releasing to the environment. Performance is measured by the intensity of the fragrance release during the pre-rub phase and post-rub. The pre-rub phase is the phase when the capsules have been deposited on the cloth, e.g., after a fabric softener containing capsules has been used during the wash cycle. The post-rub phase is after the capsules have been deposited and the capsules are broken by friction or other similar mechanisms.

In general, the amount of the capsule formation aid varies from 0.1 to 5% (e.g., 0.05 to 0.2%, 0.5 to 4%, 0.2 to 2%, 1 to 2%, and 1% to 3%) by weight of the capsule composition.

Some capsule formation aids are protective colloids or emulsifiers including maleic-vinyl copolymers such as the copolymers of vinyl ethers with maleic anhydride or acid, sodium lignosulfonates, maleic anhydride/styrene copolymers, ethylene/maleic anhydride copolymers, and copolymers of propylene oxide, ethylenediamine and ethylene oxide, polyvinylpyrrolidone, fatty acid esters of polyoxyethylenated sorbitol and sodium dodecylsulfate.

Commercially available surfactants include, but are not limited to, sulfonated naphthalene-formaldehyde condensates such as MORWET D425 (Akzo Nobel); partially hydrolyzed polyvinyl alcohols such as MOWIOLs, e.g., MOWIOL 3-83 (Air Products); ethylene oxide-propylene oxide block copolymers or poloxamers such as PLURONIC, SYNPERONIC or PLURACARE materials (BASF); sulfonated polystyrenes such as FLEXAN II (Akzo Nobel); and ethylene-maleic anhydride polymers such as ZEMAC (Vertellus Specialties Inc.). Examples of surfactants that can be used in combination with the dispersant include, but are not limited to, cetyl trimethyl ammonium chloride (CTAC), poloxamers such as PLURONICS (e.g., PLURONIC F127), PLURAFAC (e.g., PLURAFAC F127), or MIRANET-N, saponins such as QNATURALE (National Starch Food Innovation); or a gum Arabic such as Seyal or Senegal. The amount of surfactant present in the capsule slurry can vary depending on the surfactant used. In some embodiments the amount of surfactant is in the range of 0.05 to 0.2 weight percent, in particular when CTAC is employed. In another embodiment, the amount of surfactant is in the range of 1 to 3 weight percent when a saponin or gum arabic is used.

Optionally, an emulsifier (i.e., nonionic such as polyoxyethylene sorbitan monostearate (e.g., TWEEN 60), anionic such as sodium oleate, zwitterionic such as lecithins) from 0.01 weight % to 25 weight %, more preferably from 5 weight % to 10 weight % can be included.

In other embodiments, the capsule formation aid is a processing aid such as hydrocolloids, which improve the colloidal stability of the capsule suspension or slurry against coagulation, sedimentation and creaming. As such, such processing aids can also be used in conjunction with the capsules of this invention. As used herein, the term “hydrocolloid” refers to a broad class of water-dispersible polymers having anionic, cationic, zwitterionic or nonionic character. In particular embodiments, the capsule suspension includes a nonionic polymer, cationic polymer, anionic polymer, anionic surfactant, or a combination thereof.

In certain embodiments, the nonionic polymer is a polyvinylpyrrolidone (PVP), polyethylene glycol (PEG), Polyethylene oxide (PEO), or polyethylene oxide-polypropylene oxide (PEO-PPO), polyethylene oxide-polypropylene oxide-polyethylene oxide (PEO-PPO-PEO). In other embodiments, the cationic polymer is Polyquaterium-6 (polydiallyldimethylammonium chloride), Polyquatemium-11 (vinyl pyrrolidone/dimethylaminoethyl methacrylate copolymer) or Polyquaternium-47 (acrylic acid/methacrylamidopropyl trimethyl ammonium chloride/methyl acrylate terpolymer). In yet other embodiments, the anionic polymer is a polystyrene sulfonic acid, polyacrylic acid, or hyaluronic acid. In still other embodiments, the anionic surfactant is sodium laureth sulfate (SLS) or a complex ester of phosphoric acid and ethoxylated cosmetic grade oleyl alcohol (e.g., CRODAFOS 010A-SS-(RB)).

Other hydrocolloids useful in the present invention include synthetic polymers and copolymers, such as poly(vinyl pyrrolidone-co-vinyl acetate), poly(vinyl alcohol-co-vinyl acetate), poly((met)acrylic acid), poly(maleic acid), poly(alkyl(meth)acrylate-co-(meth)acrylic acid), poly(acrylic acid-co-maleic acid)copolymer, poly(alkyleneoxide), poly(vinylmethylether), poly(vinylether-co-maleic anhydride), and the like, as well as poly-(ethyleneimine), poly((meth)acrylamide), poly(alkyleneoxide-co-dimethylsiloxane), poly(amino dimethylsiloxane), and their quartenized forms.

The capsule formation aid may also be used in combination with CMC, polyvinylpyrrolidone, alkylnaphthalenesulfonate formaldehyde condensates, and/or a surfactant during processing to facilitate capsule formation. Examples of surfactants that can be used in combination with the capsule formation aid include, but are not limited to, cetyl trimethyl ammonium chloride (CTAC), poloxamers such as PLURONICS (e.g., PLURONIC F127), PLURAFAC (e.g., PLURAFAC F127), or MIRANET-N, saponins such as QNATURALE (National Starch Food Innovation); or a gum Arabic such as Seyal or Senegal. In certain embodiments, the CMC polymer has a molecular weight range between about 90,000 Daltons to 1,500,000 Daltons, preferably between about 250,000 Daltons to 750,000 Daltons and more preferably between 400,000 Daltons to 750,000 Daltons. The CMC polymer has a degree of substitution between about 0.1 to about 3, preferably between about 0.65 to about 1.4, and more preferably between about 0.8 to about 1.0. The CMC polymer is present in the capsule slurry at a level from about 0.1% to about 2% and preferably from about 0.3% to about 0.7%. in other embodiments, polyvinylpyrrolidone used in this invention is a water-soluble polymer and has a molecular weight of 1,000 to 10,000,000. Suitable polyvinylpyrrolidone are polyvinylpyrrolidone K12, K15, K17, K25, K30, K60, K90, or a mixture thereof. The amount of polyvinylpyrrolidone is 2-50%, 5-30%, or 10-25% by weight of the capsule composition. Commercially available alkylnaphthalenesulfonate formaldehyde condensates include MORWET D-425, which is a sodium salt of naphthalene sulfonate condensate by Akzo Nobel, Fort Worth, Tex.

In some embodiments, the capsules are purified by washing the capsule slurry with water until a neutral pH is achieved. The capsule suspension can be washed using any conventional method including the use of a separatory funnel, filter paper, centrifugation and the like. The capsule suspension can be washed one, two, three, four, five, six, seven, eight, nine, ten or more times until a predetermined pH, e.g., pH 7±0.5, is achieved. The pH of the purified capsules can be determined using any conventional method including pH paper, pH indicators, or a pH meter. A capsule suspension is “purified” in that it is 80%, 90%, 95%, 97%, 98% or 99% homogeneous to capsules, from which is removed unwanted impurities and/or starting materials, e.g., polyisocyanate, cross-linking agent and the like. The purification of the capsules can also include the additional step of adding a salt to the capsule suspension prior to the step of washing the capsule suspension with water. Exemplary salts of use in this step of the invention include, but are not limited to, sodium chloride, potassium chloride or bi-sulphite salts.

### Chain Termination Agents

Polymerization reactions for forming polyurea/polyurethane polymers can be terminated by adding a chain termination agent, e.g., a monofunctional amine or alcohol. Further, a chain termination agent also reacts with isocyanate groups on the surface of the capsules, thus reduced/eliminated isocyanate groups. Examples of a chain termination agent include C₁-C₂₀ primary and secondary amines, C₁-C₂₀ alcohols, C₁-C₂₀ thiols, and any combination thereof.

### Active Materials

Active materials include, but are not limited to, a fragrance, pro-fragrance, flavor, vitamin or derivative thereof, malodor counteractive agent, anti-inflammatory agent, fungicide, anesthetic, analgesic, antimicrobial active, anti-viral agent, anti-infectious agent, anti-acne agent, skin lightening agent, insect repellant, emollient, skin moisturizing agent, wrinkle control agent, UV protection agent, fabric softener active, hard surface cleaning active, skin or hair conditioning agent, insect repellant, animal repellent, vermin repellent, flame retardant, antistatic agent, nanometer to micron size inorganic solid, polymeric or elastomeric particle, and combination thereof.

Suitable fragrances include without limitation, any combination of fragrance oil, essential oil, plant extract or mixture thereof that is compatible with, and capable of being encapsulated by a polymer. Individual perfume ingredients that can be included in the capsules of this invention include fragrances containing:

i) hydrocarbons, such as, for example, 3-carene, α-pinene, β-pinene, α-terpinene, γ-terpinene, p-cymene, bisabolene, camphene, caryophyllene, cedrene, farnesene, limonene, longifolene, myrcene, ocimene, valencene, (E,Z)-1,3,5-undecatriene, styrene, and diphenylmethane;

ii) aliphatic alcohols, such as, for example, hexanol, octanol, 3-octanol, 2,6-dimethylheptanol, 2-methyl-2-heptanol, 2-methyl-2-octanol, (E)-2-hexenol, (E)- and (Z)-3-hexenol, 1-octen-3-ol, a mixture of 3,4,5,6,6-pentamethyl-3/4-hepten-2-ol and 3,5,6,6-tetramethyl-4-methyleneheptan-2-ol, (E,Z)-2,6-nonadienol, 3,7-dimethyl-7-methoxy-octan-2-ol, 9-decenol, 10-undecenol, 4-methyl-3-decen-5-ol, aliphatic aldehydes and their acetals such as for example hexanal, heptanal, octanal, nonanal, decanal, undecanal, dodecanal, tridecanal, 2-methyloctanal, 2-methylnonanal, (E)-2-hexenal, (Z)-4-heptenal, 2,6-dimethyl-5-heptenal, 10-undecenal, (E)-4-decenal, 2-dodecenal, 2,6,10-trimethyl-5,9-undecadienal, heptanal-diethylacetal, 1,1-dimethoxy-2,2,5-trimethyl-4-hexene, and citronellyl oxyacetaldehyde;

iii) aliphatic ketones and oximes thereof, such as, for example, 2-heptanone, 2-octanone, 3-octanone, 2-nonanone, 5-methyl-3-heptanone, 5-methyl-3-heptanone oxime, 2,4,4,7-tetramethyl-6-octen-3-one, aliphatic sulfur-containing compounds, such as for example 3-methylthiohexanol, 3-methylthiohexyl acetate, 3-mercaptohexanol, 3-mercaptohexyl acetate, 3-mercaptohexyl butyrate, 3-acetylthiohexyl acetate, 1-menthene-8-thiol, and aliphatic nitriles (e.g., 2-nonenenitrile, 2-tridecenenitrile, 2,12-tridecenenitrile, 3,7-dimethyl-2,6-octadienenitrile, and 3,7-dimethyl-6-octenenitrile);

iv) aliphatic carboxylic acids and esters thereof, such as, for example, (E)- and (Z)-3-hexenylformate, ethyl acetoacetate, isoamyl acetate, hexyl acetate, 3,5,5-trimethylhexyl acetate, 3-methyl-2-butenyl acetate, (E)-2-hexenyl acetate, (E)- and (Z)-3-hexenyl acetate, octyl acetate, 3-octyl acetate, 1-octen-3-yl acetate, ethyl butyrate, butyl butyrate, isoamyl butyrate, hexylbutyrate, (E)- and (Z)-3-hexenyl isobutyrate, hexyl crotonate, ethylisovalerate, ethyl-2-methyl pentanoate, ethyl hexanoate, allyl hexanoate, ethyl heptanoate, allyl heptanoate, ethyl octanoate, ethyl-(E,Z)-2,4-decadienoate, methyl-2-octinate, methyl-2-noninate, allyl-2-isoamyl oxyacetate, and methyl-3,7-dimethyl-2,6-octadienoate;

v) acyclic terpene alcohols, such as, for example, citronellol; geraniol; nerol; linalool; lavandulol; nerolidol; farnesol; tetrahydrolinalool; tetrahydrogeraniol; 2,6-dimethyl-7-octen-2-ol; 2,6-dimethyloctan-2-ol; 2-methyl-6-methylene-7-octen-2-ol; 2,6-dimethyl-5,7-octadien-2-ol; 2,6-dimethyl-3,5-octadien-2-ol; 3,7-dimethyl-4,6-octadien-3-ol; 3,7-dimethyl-1,5,7-octatrien-3-ol 2,6-dimethyl-2,5,7-octatrien-1-ol; as well as formates, acetates, propionates, isobutyrates, butyrates, isovalerates, pentanoates, hexanoates, crotonates, tiglinates and 3-methyl-2-butenoates thereof;

vi) acyclic terpene aldehydes and ketones, such as, for example, geranial, neral, citronellal, 7-hydroxy-3,7-dimethyloctanal, 7-methoxy-3,7-dimethyloctanal, 2,6,10-trimethyl-9-undecenal, α-sinensal, β-sinensal, geranylacetone, as well as the dimethyl- and diethylacetals of geranial, neral and 7-hydroxy-3,7-dimethyloctanal;

vii) cyclic terpene alcohols, such as, for example, menthol, isopulegol, alpha-terpineol, terpinen-4-ol, menthan-8-ol, menthan-1-ol, menthan-7-ol, borneol, isoborneol, linalool oxide, nopol, cedrol, ambrinol, vetiverol, guaiol, and the formates, acetates, propionates, isobutyrates, butyrates, isovalerates, pentanoates, hexanoates, crotonates, tiglinates and 3-methyl-2-butenoates of alpha-terpineol, terpinen-4-ol, methan-8-ol, methan-1-ol, methan-7-ol, borneol, isoborneol, linalool oxide, nopol, cedrol, ambrinol, vetiverol, and guaiol;

viii) cyclic terpene aldehydes and ketones, such as, for example, menthone, isomenthone, 8-mercaptomenthan-3-one, carvone, camphor, fenchone, α-ionone, β-ionone, α-n-methylionone, β-n-methylionone, α-isomethylionone, β-isomethylionone, alpha-irone, α-damascone, β-damascone, β-damascenone, δ-damascone, γ-damascone, 1-(2,4,4-trimethyl-2-cyclohexen-1-yl)-2-buten-1-one, 1,3,4,6,7,8a-hexahydro-1,1,5,5-tetramethyl-2H-2,4a-methanonaphthalen-8(5H-)-one, nootkatone, dihydronootkatone; acetylated cedarwood oil (cedryl methyl ketone);

ix) cyclic alcohols, such as, for example, 4-tert-butylcyclohexanol, 3,3,5-trimethylcyclohexanol, 3-isocamphylcyclohexanol, 2,6,9-trimethyl-Z2,Z5,E9-cyclodode-catrien-1-ol, 2-isobutyl-4-methyltetrahydro-2H-pyran-4-ol;

x) cycloaliphatic alcohols, such as, for example, alpha, 3,3-trimethylcyclo-hexylmethanol, 2-methyl-4-(2,2,3-trimethyl-3-cyclopent-1-yl)butanol, 2-methyl-4-(2,2,3-trimethyl-3-cyclopent-1-yl)-2-buten-1-ol, 2-ethyl-4-(2,2,3-trimethyl-3-cyclopent-1-yl)-2-buten-1-ol, 3-methyl-5-(2,2,3-trimethyl-3-cyclopent-1-yl)-pentan-2-ol, 3-methyl-5-(2,2,3-trimethyl-3-cyclopent-1-yl)-4-penten-2-ol, 3,3-dimethyl-5-(2,2,3-trimethyl-3-cyclopent-1-yl)-4-penten-2-ol, 1-(2,2,6-trimethylcyclohexyl)pentan-3-ol, 1-(2,2,6-trimethylcyclohexyl)hexan-3-ol;

xi) cyclic and cycloaliphatic ethers, such as, for example, cineole, cedryl methyl ether, cyclododecyl methyl ether;

xii) (ethoxymethoxy)cyclododecane; alpha-cedrene epoxide, 3a,6,6,9a-tetramethyldodecahydronaphtho[2,1-b]furan, 3a-ethyl-6,6,9a-trimethyldodecahydro-naphtho[2,1-b]furan, 1,5,9-trimethyl-13-oxabicyclo[10.1.0]-trideca-4,8-diene, rose oxide, 2-(2,4-dimethyl-3-cyclohexen-1-yl)-5-methyl-5-(1-methylpropyl)-1,3-dioxan;

xiii) cyclic ketones, such as, for example, 4-tert-butylcyclohexanone, 2,2,5-trimethyl-5-pentylcyclopentanone, 2-heptylcyclopentanone, 2-pentylcyclopentanone, 2-hydroxy-3-methyl-2-cyclopenten-1-one, 3-methyl-cis-2-penten-1-yl-2-cyclopenten-1-one, 3-methyl-2-pentyl-2-cyclopenten-1-one, 3-methyl-4-cyclopentadecenone, 3-methyl-1-cyclopentadecenone, 3-methylcyclopentadecanone, 4-(1-ethoxyvinyl)-3,3,5,5-tetra-methylcyclohexanone, 4-tert-pentylcyclohexanone, 5-cyclohexadecen-1-one, 6,7-dihydro-1,1,2,3,3-pentamethyl-4(5H)-indanone, 5-cyclohexadecen-1-one, 8-cyclohexadecen-1-one, 9-cycloheptadecen-1-one, cyclopentadecanone, cycloaliphatic aldehydes, such as, for example, 2,4-dimethyl-3-cyclohexene carbaldehyde, 2-methyl-4-(2,2,6-trimethyl-cyclohexen-1-yl)-2-butenal, 4-(4-hydroxy-4-methylpentyl)-3-cyclohexene carbaldehyde, 4-(4-methyl-3-penten-1-yl)-3-cyclohexene carbaldehyde;

xiv) cycloaliphatic ketones, such as, for example, 1-(3,3-dimethylcyclohexyl)-4-penten-1-one, 1-(5,5-dimethyl-1-cyclohexen-1-yl)-4-penten-1-one, 2,3,8,8-tetramethyl-1,2,3,4,5,6,7,8-octahydro-2-naphtalenyl methyl-ketone, methyl-2,6,10-trimethyl-2,5,9-cyclododecatrienyl ketone, tert-butyl-(2,4-dimethyl-3-cyclohexen-1-yl)ketone;

xv) esters of cyclic alcohols, such as, for example, 2-tert-butylcyclohexyl acetate, 4-tert-butylcyclohexyl acetate, 2-tert-pentylcyclohexyl acetate, 4-tert-pentylcyclohexyl acetate, decahydro-2-naphthyl acetate, 3-pentyltetrahydro-2H-pyran-4-yl acetate, decahydro-2,5,5,8a-tetramethyl-2-naphthyl acetate, 4,7-methano-3a,4,5,6,7,7a-hexahydro-5 or 6-indenyl acetate, 4,7-methano-3a,4,5,6,7,7a-hexahydro-5 or 6-indenyl propionate, 4,7-methano-3a,4,5,6,7,7a-hexahydro-5 or 6-indenyl-isobutyrate, 4,7-methanooctahydro-5 or 6-indenyl acetate;

xvi) esters of cycloaliphatic carboxylic acids, such as, for example, allyl 3-cyclohexyl-propionate, allyl cyclohexyl oxyacetate, methyl dihydrojasmonate, methyl jasmonate, methyl 2-hexyl-3-oxocyclopentanecarboxylate, ethyl 2-ethyl-6,6-dimethyl-2-cyclohexenecarboxylate, ethyl 2,3,6,6-tetramethyl-2-cyclohexenecarboxylate, ethyl 2-methyl-1, 3-dioxolane-2-acetate;

xvii) aromatic and aliphatic alcohols, such as, for example, benzyl alcohol, 1-phenylethyl alcohol, 2-phenylethyl alcohol, 3-phenylpropanol, 2-phenylpropanol, 2-phenoxyethanol, 2,2-dimethyl-3-phenylpropanol, 2,2-dimethyl-3-(3-methylphenyl)-propanol, 1,1-dimethyl-2-phenylethyl alcohol, 1,1-dimethyl-3-phenylpropanol, 1-ethyl-1-methyl-3-phenylpropanol, 2-methyl-5-phenylpentanol, 3-methyl-5-phenylpentanol, 3-phenyl-2-propen-1-ol, 4-methoxybenzyl alcohol, 1-(4-isopropylphenyl)ethanol;

xviii) esters of aliphatic alcohols and aliphatic carboxylic acids, such as, for example, benzyl acetate, benzyl propionate, benzyl isobutyrate, benzyl isovalerate, 2-phenylethyl acetate, 2-phenylethyl propionate, 2-phenylethyl isobutyrate, 2-phenylethyl isovalerate, 1-phenylethyl acetate, α-trichloromethylbenzyl acetate, α,α-dimethylphenylethyl acetate, alpha, alpha-dimethylphenylethyl butyrate, cinnamyl acetate, 2-phenoxyethyl isobutyrate, 4-methoxybenzyl acetate, araliphatic ethers, such as for example 2-phenylethyl methyl ether, 2-phenylethyl isoamyl ether, 2-phenylethyl-1-ethoxyethyl ether, phenylacetaldehyde dimethyl acetal, phenylacetaldehyde diethyl acetal, hydratropaaldehyde dimethyl acetal, phenylacetaldehyde glycerol acetal, 2,4,6-trimethyl-4-phenyl-1,3-dioxane, 4,4a,5,9b-tetrahydroindeno[1,2-d]-m-dioxin, 4,4a,5,9b-tetrahydro-2,4-dimethylindeno[1,2-d]-m-dioxin;

xix) aromatic and aliphatic aldehydes, such as, for example, benzaldehyde; phenylacetaldehyde, 3-phenylpropanal, hydratropaldehyde, 4-methylbenzaldehyde, 4-methylphenylacetaldehyde, 3-(4-ethylphenyl)-2,2-dimethylpropanal, 2-methyl-3-(4-iso-propylphenyl)propanal, 2-methyl-3-(4-tert-butylphenyl)propanal, 3-(4-tert-butylphenyl)-propanal, cinnamaldehyde, alpha-butylcinnamaldehyde, alpha-amylcinnamaldehyde, alpha-hexylcinnamaldehyde, 3-methyl-5-phenylpentanal, 4-methoxybenzaldehyde, 4-hydroxy-3-methoxybenzaldehyde, 4-hydroxy-3-ethoxybenzaldehyde, 3,4-methylene-dioxybenzaldehyde, 3,4-dimethoxybenzaldehyde, 2-methyl-3-(4-methoxyphenyl)-propanal, 2-methyl-3-(4-methylendioxyphenyl)propanal;

xx) aromatic and aliphatic ketones, such as, for example, acetophenone, 4-methylacetophenone, 4-methoxyacetophenone, 4-tert-butyl-2,6-dimethylacetophenone, 4-phenyl-2-butanone, 4-(4-hydroxyphenyl)-2-butanone, 1-(2-naphthalenyl)ethanone, benzophenone, 1,1,2,3,3,6-hexamethyl-5-indanyl methyl ketone, 6-tert-butyl-1,1-dimethyl-4-indanyl methyl ketone, 1-[2,3-dihydro-1,1,2,6-tetramethyl-3-(1-methylethyl)-1H-5-indenyl]ethanone, 5′,6′,7′,8′-tetrahydro-3′,5′,5′,6′,8′,8′-hexamethyl-2-aceto-naphthone;

xxi) aromatic and araliphatic carboxylic acids and esters thereof, such as, for example, benzoic acid, phenylacetic acid, methyl benzoate, ethyl benzoate, hexyl benzoate, benzyl benzoate, methyl phenylacetate, ethyl phenylacetate, geranyl phenylacetate, phenylethyl phenylacetate, methyl cinnamate, ethyl cinnamate, benzyl cinnamate, phenylethyl cinnamate, cinnamyl cinnamate, allyl phenoxyacetate, methyl salicylate, isoamyl salicylate, hexyl salicylate, cyclohexyl salicylate, cis-3-hexenyl salicylate, benzyl salicylate, phenylethyl salicylate, methyl 2,4-dihydroxy-3,6-dimethylbenzoate, ethyl 3-phenylglycidate, ethyl 3-methyl-3-phenylglycidate;

xxii) nitrogen-containing aromatic compounds, such as, for example, 2,4,6-trinitro-1,3-dimethyl-5-tert-butylbenzene, 3,5-dinitro-2,6-dimethyl-4-tert-butylaceto-phenone, cinnamonitrile, 5-phenyl-3-methyl-2-pentenonitrile, 5-phenyl-3-methylpentano-nitrite, methyl anthranilate, methy-N-methylanthranilate, Schiff's bases of methyl anthranilate with 7-hydroxy-3,7-dimethyloctanal, 2-methyl-3-(4-tert-butylphenyl)-propanal or 2,4-dimethyl-3-cyclohexene carbaldehyde, 6-isopropylquinoline, 6-isobutyl-quinoline, 6-sec-butylquinoline, indole, skatole, 2-methoxy-3-isopropylpyrazine, 2-isobutyl-3-methoxypyrazine;

xxiii) phenols, phenyl ethers and phenyl esters, such as, for example, estragole, anethole, eugenol, eugenyl methyl ether, isoeugenol, isoeugenol methyl ether, thymol, carvacrol, diphenyl ether, beta-naphthyl methyl ether, beta-naphthyl ethyl ether, beta-naphthyl isobutyl ether, 1,4-dimethoxybenzene, eugenyl acetate, 2-methoxy-4-methylphenol, 2-ethoxy-5-(1-propenyl)phenol, p-cresyl phenylacetate;

xxiv) heterocyclic compounds, such as, for example, 2,5-dimethyl-4-hydroxy-2H-furan-3-one, 2-ethyl-4-hydroxy-5-methyl-2H-furan-3-one, 3-hydroxy-2-methyl-4H-pyran-4-one, 2-ethyl-3-hydroxy-4H-pyran-4-one;

xxv) lactones, such as, for example, 1,4-octanolide, 3-methyl-1,4-octanolide, 1,4-nonanolide, 1,4-decanolide, 8-decen-1,4-olide, 1,4-undecanolide, 1,4-dodecanolide, 1,5-decanolide, 1,5-dodecanolide, 1,15-pentadecanolide, cis- and trans-11-pentadecen-1,15-olide, cis- and trans-12-pentadecen-1,15-olide, 1,16-hexadecanolide, 9-hexadecen-1,16-olide, 10-oxa-1,16-hexadecanolide, 11-oxa-1,16-hexadecanolide, 12-oxa-1,16-hexadecanolide, ethylene-1,12-dodecanedioate, ethylene-1,13-tridecanedioate, coumarin, 2,3-dihydrocoumarin, and octahydrocoumarin; and

xxvi) essential oils, concretes, absolutes, resins, resinoids, balsams, tinctures such as for example ambergris tincture, amyris oil, angelica seed oil, angelica root oil, aniseed oil, valerian oil, basil oil, tree moss absolute, bay oil, armoise oil, benzoe resinoid, bergamot oil, beeswax absolute, birch tar oil, bitter almond oil, savory oil, buchu leaf oil, cabreuva oil, cade oil, calamus oil, camphor oil, cananga oil, cardamom oil, cascarilla oil, cassia oil, cassie absolute, castoreum absolute, cedar leaf oil, cedar wood oil, cistus oil, citronella oil, lemon oil, copaiba balsam, copaiba balsam oil, coriander oil, costus root oil, cumin oil, cypress oil, davana oil, dill weed oil, dill seed oil, eau de brouts absolute, oakmoss absolute, elemi oil, estragon oil, eucalyptus citriodora oil, eucalyptus oil (cineole type), fennel oil, fir needle oil, galbanum oil, galbanum resin, geranium oil, grapefruit oil, guaiacwood oil, gurjun balsam, gurjun balsam oil, helichrysum absolute, helichrysum oil, ginger oil, iris root absolute, iris root oil, jasmine absolute, calamus oil, blue camomile oil, Roman camomile oil, carrot seed oil, cascarilla oil, pine needle oil, spearmint oil, caraway oil, labdanum oil, labdanum absolute, labdanum resin, lavandin absolute, lavandin oil, lavender absolute, lavender oil, lemon-grass oil, lovage oil, lime oil distilled, lime oil expressed, linaloe oil, Litsea cubeba oil, laurel leaf oil, mace oil, marjoram oil, mandarin oil, massoi (bark) oil, mimosa absolute, ambrette seed oil, musk tincture, clary sage oil, nutmeg oil, myrrh absolute, myrrh oil, myrtle oil, clove leaf oil, clove bud oil, neroli oil, olibanum absolute, olibanum oil, opopanax oil, orange flower absolute, orange oil, origanum oil, palmarosa oil, patchouli oil, perilla oil, Peru balsam oil, parsley leaf oil, parsley seed oil, petitgrain oil, peppermint oil, pepper oil, pimento oil, pine oil, pennyroyal oil, rose absolute, rosewood oil, rose oil, rosemary oil, Dalmatian sage oil, Spanish sage oil, sandal-wood oil, celery seed oil: spike-lavender oil, star anise oil, storax oil, tagetes oil, fir needle oil, tea tree oil, turpentine oil, thyme oil, Tolu balsam, tonka bean absolute, tuberose absolute, vanilla extract, violet leaf absolute, verbena oil, vetiver oil, juniperberry oil, wine lees oil, wormwood oil, wintergreen oil, ylang-ylang oil, hyssop oil, civet absolute, cinnamon leaf oil, cinnamon bark oil, and fractions thereof or ingredients isolated therefrom;

(xxvii) flavors including, but are not limited to, acetaldehyde, dimethyl sulfide, ethyl acetate, ethyl propionate, methyl butyrate, and ethyl butyrate. Flavors containing volatile aldehydes or esters include, e.g., cinnamyl acetate, cinnamaldehyde, citral, diethylacetal, dihydrocarvyl acetate, eugenyl formate, and p-methylanisole. Further examples of volatile compounds that may be present in the instant flavor oils include acetaldehyde (apple); benzaldehyde (cherry, almond); cinnamic aldehyde (cinnamon); citral, i.e., alpha citral (lemon, lime); neral, i.e., beta citral (lemon, lime); decanal (orange, lemon); ethyl vanillin (vanilla, cream); heliotropine, i.e., piperonal (vanilla, cream); vanillin (vanilla, cream); alpha-amyl cinnamaldehyde (spicy fruity flavors); butyraldehyde (butter, cheese); valeraldehyde (butter, cheese); citronellal (modifies, many types); decanal (citrus fruits); aldehyde C-8 (citrus fruits); aldehyde C-9 (citrus fruits); aldehyde C-12 (citrus fruits); 2-ethyl butyraldehyde (berry fruits); hexenal, i.e., trans-2 (berry fruits); tolyl aldehyde (cherry, almond); veratraldehyde (vanilla); 2,6-dimethyl-5-heptenal, i.e., melonal (melon); 2-6-dimethyloctanal (green fruit); and 2-dodecenal (citrus, mandarin); cherry; or grape and mixtures thereof. The composition may also contain taste modulators and artificial sweeteners. As used herein, flavor is understood to include spice oleoresins derived from allspice, basil, capsicum, cinnamon, cloves, cumin, dill, garlic, marjoram, nutmeg, paprika, black pepper, rosemary, and turmeric, essential oils, anise oil, caraway oil, clove oil, eucalyptus oil, fennel oil, garlic oil, ginger oil, peppermint oil, onion oil, pepper oil, rosemary oil, spearmint oil, citrus oil, orange oil, lemon oil, bitter orange oil, tangerine oil, alliaceous flavors, garlic, leek, chive, and onion, botanical extracts, arnica flower extract, chamomile flower extract, hops extract, marigold extract, botanical flavor extracts, blackberry, chicory root, cocoa, coffee, kola, licorice root, rose hips, sarsaparilla root, sassafras bark, tamarind and vanilla extracts, protein hydrolysates, hydrolyzed vegetable proteins, meat protein hydrolyzes, milk protein hydrolyzates and compounded flavors both natural and artificial including those disclosed in S. Heath, Source Book of Flavors, Avi Publishing Co., Westport Conn., 1981, pages 149-277. Specific preferred flavor adjuvants include, but are not limited to, the following: anise oil; ethyl-2-methyl butyrate; vanillin; cis-3-heptenol; cis-3-hexenol; trans-2-heptenal; butyl valerate; 2,3-diethyl pyrazine; methylcyclo-pentenolone; benzaldehyde; valerian oil; 3,4-dimeth-oxyphenol; amyl acetate; amyl cinnamate, y-butyryl lactone; furfural; trimethyl pyrazine; phenyl acetic acid; isovaleraldehyde; ethyl maltol; ethyl vanillin; ethyl valerate; ethyl butyrate; cocoa extract; coffee extract; peppermint oil; spearmint oil; clove oil; anethol; cardamom oil; wintergreen oil; cinnamic aldehyde; ethyl-2-methyl valerate; g-hexenyl lactone; 2,4-decadienal; 2,4-heptadienal; methyl thiazole alcohol (4-methyl-5-b-hydroxyethyl thiazole); 2-methyl butanethiol; 4-mercapto-2-butanone; 3-mercapto-2-pentanone; 1-mercapto-2-propane; benzaldehyde; furfural; furfuryl alcohol; 2-mercapto propionic acid; alkyl pyrazine; methyl pyrazine; 2-ethyl-3-methyl pyrazine; tetramethyl pyrazine; polysulfides; dipropyl disulfide; methyl benzyl disulfide; alkyl thiophene; 2,3-dimethyl thiophene; 5-methyl furfural; acetyl furan; 2,4-decadienal; guiacol; phenyl acetaldehyde; b-decalactone; d-limonene; acetoin; amyl acetate; maltol; ethyl butyrate; levulinic acid; piperonal; ethyl acetate; n-octanal; n-pentanal; n-hexanal; diacetyl; monosodium glutamate; monopotassium glutamate; sulfur-containing amino acids, e.g., cysteine; hydrolyzed vegetable protein; 2-methylfuran-3-thiol; 2-methyldihydrofuran-3-thiol; 2,5-dimethylfuran-3-thiol; hydrolyzed fish protein; tetramethyl pyrazine; propylpropenyl disulfide; propylpropenyl trisulfide; diallyl disulfide; diallyl trisulfide; dipropenyl disulfide; dipropenyl trisulfide; 4-methyl-2-[(methylthio)-ethyl]-1,3-dithiolane; 4,5-dimethyl-2-(methylthiomethyl)-1,3-dithiolane; 4-methyl-2-(methylthiomethyl)-1,3-dithiolane, and the flavor ingredients described in U.S. Pat. Nos. 6,110,520 and 6,333,180;

(xxviii) taste masking agents, substances for masking one or more unpleasant taste sensations, in particular a bitter, astringent and/or metallic taste sensation or aftertaste. Examples include lactisol [2O-(4-methoxyphenyl) lactic acid] (cf U.S. Pat. No. 5,045,336), 2,4-dihydroxybenzoic acid potassium salt (cf U.S. Pat. No. 5,643,941), ginger extracts (cf GB 2,380,936), neohesperidine dihydrochalcone (cf Manufacturing Chemist 2000, July issue, p. 16-17), specific flavones (2-phenylchrom-2-en-4-ones) (cf U.S. Pat. No. 5,580,545), specific nucleotides, for example cytidine-5′-monophosphates (CMP) (cf US 2002/0177576), specific sodium salts, such as sodium chloride, sodium citrate, sodium acetate and sodium lactate (cf Nature, 1997, Vol. 387, p. 563), a lipoprotein of .beta.-lactoglobulin and phosphatidic acid (cf EPA 635 218), neodiosmine [5,7-dihydroxy-2-(4-methoxy-3-hydroxyphenyl)-7-O-neohesperidosyl-chrom-2-en-4-one] (cf U.S. Pat. No. 4,154,862), preferably hydroxyflavanones according to EP 1 258 200, in turn preferred in this respect 2-(4-hydroxyphenyl)-5,7-dihydroxychroman-4-one (naringenin), 2-(3,4-dihydroxyphenyl)-5,7-dihydroxychroman-4-one (eriodictyol), 2-(3,4-dihydroxyphenyl)-5-hydroxy-7-methoxychroman-4-one (eriodictyol-7-methylether), 2-(3,4-dihydroxyphenyl)-7-hydroxy-5-methoxychroman-4-one (eriodictyol-5-methylether) and 2-(4-hydroxy-3-methoxyphenyl)-5,7-dihydroxychroman-4-one (homoeriodictyol), the (2S)- or (2R)-enantiomers thereof or mixtures thereof as well as the mono- or polyvalent phenolate salts thereof with Na⁺, K⁺, NH4⁺, Ca²⁺, Mg²⁺ or Al³⁺ as counter cations or .gamma.-aminobutyric acid (4-aminobutyric acid, as the neutral form (“inner salt”) or in the carboxylate or ammonium form) according to WO 2005/09684;

(xxix) taste sensates including hot tasting, salivation-inducing substances, substances causing a warmth or tingling feeling, and cooling active ingredients. Examples of hot tasting and/or salivation-inducing substances and/or substances which cause a feeling of warmth and/or a tingling feeling on the skin or on the mucous membranes and which can be a constituent of the products according to the invention are: capsaicin, dihydrocapsaicin, gingerol, paradol, shogaol, piperine, carboxylic acid-N-vanillylamides, in particular nonanoic acid-N-vanillylamide, pellitorin or spilanthol, 2-nonanoic acid amides, in particular 2-nonanoic acid-N-isobutylamide, 2-nonanoic acid-N-4-hydroxy-3-methoxyphenylamide, alkyl ethers of 4-hydroxy-3-methoxybenzyl alcohol, in particular 4-hydroxy-3-methoxybenzyl-n-butylether, alkyl ethers of 4-acyloxy-3-methoxybenzyl alcohol, in particular 4-acetyloxy-3-methoxybenzyl-n-butylether and 4-acetyloxy-3-methoxybenzyl-n-hexylether, alkyl ethers of 3-hydroxy-4-methoxybenzyl alcohol, alkyl ethers of 3,4-dimethoxybenzyl alcohol, alkyl ethers of 3-ethoxy-4-hydroxybenzyl alcohol, alkyl ethers of 3,4-methylene dioxybenzyl alcohol, (4-hydroxy-3-methoxyphenyl)acetic acid amides, in particular (4-hydroxy-3-methoxyphenyl)acetic acid-N-n-octylamide, vanillomandelic acid alkylamides, ferulic acid-phenethylamides, nicotinaldehyde, methylnicotinate, propylnicotinate, 2-butoxyethylnicotinate, benzylnicotinate, 1-acetoxychavicol, polygodial and isodrimeninol, further preferred cis- and/or trans-pellitorin according to WO 2004/000787 or WO 2004/043906, alkenecarboxylic acid-N-alkylamides according to WO 2005/044778, mandelic acid alkylamides according to WO 03/106404 or alkyloxyalkanoic acid amides according to WO 2006/003210. Examples of preferred hot tasting natural extracts and/or natural extracts which cause a feeling of warmth and/or a tingling feeling on the skin or on the mucous membranes and which can be a constituent of the products according to the invention are: extracts of paprika, extracts of pepper (for example capsicum extract), extracts of chili pepper, extracts of ginger roots, extracts of Aframomum melgueta, extracts of Spilanthes-acmella, extracts of Kaempferia galangal or extracts of Alpinia galanga. Suitable cooling active ingredients include the following: 1-menthol, d-menthol, racemic menthol, menthone glycerol acetal (trade name: Frescolat®MGA), menthyl lactate (trade name: Frescolat®ML, menthyl lactate preferably being 1-menthyl lactate, in particular 1-menthyl-1-lactate), substituted menthyl-3-carboxamides (for example menthyl-3-carboxylic acid-N-ethylamide), 2-isopropyl-N-2,3-trimethyl-butanamide, substituted cyclohexane carboxamides, 3-menthoxypropane-1,2-diol, 2-hydroxyethyl menthyl carbonate, 2-hydroxypropyl menthyl carbonate, N-acetylglycine menthyl ester, isopulegol, hydroxycarboxylic acid menthyl esters (for example menthyl-3-hydroxybutyrate), monomenthyl succinate, 2-mercaptocyclo-decanone, menthyl-2-pyrrolidin-5-onecarboxylate, 2,3-dihydroxy-p-menthane, 3,3,5-trimethylcyclohexanone glycerol ketal, 3-menthyl-3,6-di- and -trioxaalkanoates, 3-menthyl methoxyacetate and icilin. Cooling active ingredients which are particularly preferred are as follows: 1-menthol, racemic menthol, menthone glycerol acetal (trade name: Frescolat®MGA), menthyl lactate (preferably 1-menthyl lactate, in particular 1-menthyl-1-lactate, trade name: Frescolat®ML), 3-menthoxypropane-1,2-diol, 2-hydroxyethyl menthyl carbonate, 2-hydroxypropyl menthyl carbonate.

(xxx) malodor counteracting agents including an α,β-unsaturated carbonyl compounds including but not limited to those disclosed in U.S. Pat. No. 6,610,648 and EP 2,524,704, amyl cinnamaldehyde, benzophenone, benzyl benzoate, benzyl isoeugenol, benzyl phenyl acetate, benzyl salicylate, butyl cinnamate, cinnamyl butyrate, cinnamyl isovalerate, cinnamyl propionate, decyl acetate, ethyl myristate, isobutyl cinnamate, isoamyl salicylate, phenethyl benzoate, phenethyl phenyl acetate, triethyl citrate, tripropylene glycol n-butyl ether, isomers of bicyclo[2.2.1]hept-5-ene-2-carboxylic acid, ethyl ester, nano silver, zinc undecenylate, β-naphthyl methyl ether, β-naphthyl ketone, benzyl acetone. They may include mixture of hexahydro-4,7-methanoinden-5-yl propionate and hexahydro-4,7-methanoinden-6-yl propionate; 4-(2,6,6-trimethyl-2-cyclohexen-1-yl)-3-methyl-3-buten-2-one; 3,7-dimethyl-2,6-nonadien-1-nitrile; dodeca-hydro-3a,6,6,9a-tetramethylnaphtho(2,1-b)furan; ethylene glycol cyclic ester of n-dodecanedioic acid; 1-cyclohexadecen-6-one; 1-cycloheptadecen-10-one; and corn mint oil. They may also include 1-cyclohexylethan-1-yl butyrate; 1-cyclohexylethan-1-yl acetate; 1-cyclohexylethan-1-ol; 1-(4′-methylethyl)cyclohexylethan-1-yl propionate; and 2′-hydroxy-1′-ethyl(2-phenoxy)acetate each of which compound is marketed under the trademark VEILEX by International Flavors & Fragrances Inc. More suitable malodor counteracting agents are polymers containing an α-keto, benzaldehyde, or α,β-unsaturated carbonyl moiety, such as those described in US Application Publications 2012/0294821, 2013/0101544 and 2013/0101545;

(xxxi) vitamins including any vitamin, a derivative thereof and a salt thereof. Examples are as follows: vitamin A and its analogs and derivatives (e.g., retinol, retinal, retinyl palmitate, retinoic acid, tretinoin, and iso-tretinoin, known collectively as retinoids), vitamin E (tocopherol and its derivatives), vitamin C (L-ascorbic acid and its esters and other derivatives), vitamin B3 (niacinamide and its derivatives), alpha hydroxy acids (such as glycolic acid, lactic acid, tartaric acid, malic acid, citric acid, etc.) and beta hydroxy acids (such as salicylic acid and the like);

(xxxii) antibacterials including bisguanidines (e.g., chlorhexidine digluconate), diphenyl compounds, benzyl alcohols, trihalocarbanilides, quaternary ammonium compounds, ethoxylated phenols, and phenolic compounds, such as halo-substituted phenolic compounds, like PCMX (i.e., p-chloro-m-xylenol), triclosan (i.e., 2,4,4′-trichloro-2′hydroxy-diphenylether), thymol, and triclocarban;

(xxxiii) sunscreen actives including oxybenzone, octylmethoxy cinnamate, butylmethoxy dibenzoyln ethane, p-aminobenzoic acid and octyl dimethyl-p-aminobenzoic acid;

(xxxiv) antioxidants such as beta-carotene, vitamin C (Ascorbic Acid) or an ester thereof, vitamin A or an ester thereof, vitamin E or an ester thereof, lutein or an ester thereof, lignan, lycopene, selenium, flavonoids, vitamin-like antioxidants such as coenzyme Q10 (CoQ10) and glutathione, and antioxidant enzymes such as superoxide dismutase (SOD), catalase, and glutathione peroxidase;

(xxxv) anti-inflammatory agents including, e.g., methyl salicylate, aspirin, ibuprofen, and naproxen. Additional anti-inflammatories useful in topical applications include corticosteroids, such as, but not limited to, flurandrenolide, clobetasol propionate, halobetasol propionate, fluticasone propionate, betamethasone dipropionate, betamethasone benzoate, betamethasone valerate, desoximethasone, dexamethasone, diflorasone diacetate, mometasone furoate, amcinodine, halcinonide, fluocinonide, fluocinolone acetonide, desonide, triamcinolone acetonide, hydrocortisone, hydrocortisone acetate, fluoromethalone, methylprednisolone, and predinicarbate;

(xxxvi) anesthetics that can be delivered locally including benzocaine, butamben, butamben picrate, cocaine, procaine, tetracaine, lidocaine and pramoxine hydrochloride;

(xxxvii) analgesics such as ibuprofen, diclofenac, capsaicin, and lidocaine;

(xxxviii) antifungal agents. Non-limiting examples are micanazole, clotrimazole, butoconazole, fenticonasole, tioconazole, terconazole, sulconazole, fluconazole, haloprogin, ketonazole, ketoconazole, oxinazole, econazole, itraconazole, torbinafine, nystatin and griseofulvin;

(xxxix) antibiotics such as erythromycin, clindamycin, synthomycin, tetracycline, metronidazole and the like;

(xl) anti-viral agents including famcyclovir, valacyclovir and acyclovir;

(xli) anti-parasitic agents such as scabicedes, such as permethrin, crotamiton, lindane and ivermectin;

(xlii) anti-infectious and anti-acne agents including benzoyl peroxide, sulfur, resorcinol and salicylic acid;

(xliii) dermatological active ingredients useful in topical applications including, e.g., jojoba oil and aromatic oils such as methyl salicylate, wintergreen, peppermint oil, bay oil, eucalyptus oil and citrus oils, as well as ammonium phenolsulfonate, bismuth subgallate, zinc phenolsulfonate and zinc salicylate;

(xliv) enzymes and co-enzymes useful for topical application including coenzyme Q10, papain enzyme, lipases, proteases, superoxide dismutase, fibrinolysin, desoxyribonuclease, trypsin, collagenase and sutilains;

(xlv) skin whitening agents such as hydroquinone and monobenzone;

(xlvi) anti-histamines including chlorpheniramine, brompheniramine, dexchlorpheniramine, tripolidine, clemastine, diphenhydramine, prometazine, piperazines, piperidines, astemizole, loratadine and terfonadine;

(xlvii) chemotherapeutic agents such as 5-fluorouracil, masoprocol, mechlorethamine, cyclophosphamide, vincristine, chlorambucil, streptozocin, methotrexate, bleomycin, dactinomycin, daunorubicin, coxorubicin and tamoxifen; and

(xlviii) insect repellents including pediculicides for treatment of lice, such as pyrethrins, permethrin, malathion, lindane and the like.

In addition to the active materials listed above, the products of this invention can also contain, for example, the following dyes, colorants or pigments: lactoflavin (riboflavin), beta-carotene, riboflavin-5′-phosphate, alpha-carotene, gamma-carotene, cantaxanthin, erythrosine, curcumin, quinoline yellow, yellow orange S, tartrazine, bixin, norbixin (annatto, orlean), capsanthin, capsorubin, lycopene, beta-apo-8′-carotenal, beta-apo-8′-carotenic acid ethyl ester, xantophylls (flavoxanthin, lutein, cryptoxanthin, rubixanthin, violaxanthin, rodoxanthin), fast carmine (carminic acid, cochineal), azorubin, cochineal red A (Ponceau 4 R), beetroot red, betanin, anthocyanins, amaranth, patent blue V, indigotine I (indigo-carmine), chlorophylls, copper compounds of chlorophylls, acid brilliant green BS (lissamine green), brilliant black BN, vegetable carbon, titanium dioxide, iron oxides and hydroxides, calcium carbonate, aluminum, silver, gold, pigment rubine BK (lithol rubine BK), methyl violet B, victoria blue R, victoria blue B, acilan brilliant blue FFR (brilliant wool blue FFR), naphthol green B, acilan fast green 10 G (alkali fast green 10 G), ceres yellow GRN, sudan blue II, ultramarine, phthalocyanine blue, phthalocayanine green, fast acid violet R. Further naturally obtained extracts (for example paprika extract, black carrot extract, red cabbage extract) can be used for coloring purposes. Goods results are also achieved with the colors named in the following, the so-called aluminum lakes: FD & C Yellow 5 Lake, FD & C Blue 2 Lake, FD & C Blue 1 Lake, Tartrazine Lake, Quinoline Yellow Lake, FD & C Yellow 6 Lake, FD & C Red 40 Lake, Sunset Yellow Lake, Carmoisine Lake, Amaranth Lake, Ponceau 4R Lake, Erythrosyne Lake, Red 2G Lake, Allura Red Lake, Patent Blue V Lake, Indigo Carmine Lake, Brilliant Blue Lake, Brown HT Lake, Black PN Lake, Green S Lake and mixtures thereof.

Flavor oils may contain the following solvents/diluents: ethanol, vegetable oil triglycerides, 1,2-propylene glycol, benzyl alcohol, triacetin (glycerol triacetate), diacetin (glycerol diacetate), triethyl citrate, glycerol.

When the active material is a fragrance, it is preferred that fragrance ingredients within a fragrance having a C log P of 0.5 to 15 are employed. For instance, the ingredients having a C log P value between 0.5 to 8 (e.g., between 1 to 12, between 1.5 to 8, between 2 and 7, between 1 and 6, between 2 and 6, between 2 and 5, between 3 and 7) are 25% or greater (e.g., 50% or greater and 90% or greater) by the weight of the fragrance.

In some embodiments, it is preferred that a fragrance having a weight-averaged C log P of 2.5 and greater (e.g., 3 or greater, 2.5 to 7, and 2.5 to 5) is employed. The weight-averaged C log P is calculated as follows:

C log P={Sum[(Wi)(C log P)i]}/{Sum Wi},

in which Wi is the weight fraction of each fragrance ingredient and (C log P)i is the C log P of that fragrance ingredient.

As an illustration, it is preferred that greater than 60 weight percent, preferably greater than 80 and more preferably greater than 90 weight percent of the fragrance chemicals have C log P values of greater than 2, preferably greater than 3.3, more preferably greater than 4, and even more preferably greater than 4.5.

In other embodiments, the ingredients having a C log P value between 2 and 7 (e.g., between 2 and 6, and between 2 and 5) are 25% or greater (e.g., 50% or greater and 90% or greater) by the weight of the fragrance. In still other embodiments, it is preferred that greater than 60%, preferably greater than 80% and more preferably greater than 90% of the fragrance chemicals have Clog P values of greater than 3.3, preferably greater than 4 and most preferably greater than 4.5.

Those with skill in the art will appreciate that many fragrances can be created employing various solvents and fragrance chemicals. The use of a relatively low to intermediate C log P fragrance ingredients will result in fragrances that are suitable for encapsulation. These fragrances are generally water-insoluble, to be delivered through the capsule systems of this invention onto consumer products in different stages such as damp and dry fabric. Without encapsulation, the free fragrances would normally have evaporated or dissolved in water during use, e.g., wash. Though high log P materials are generally well delivered from a regular (non-encapsulated) fragrance in a consumer product, they have excellent encapsulation properties and are also suitable for encapsulation for overall fragrance character purposes, very long-lasting fragrance delivery, or overcoming incompatibility with the consumer product, e.g., fragrance materials that would otherwise be instable, cause thickening or discoloration of the product or otherwise negatively affect desired consumer product properties.

In some embodiments, the amount of encapsulated active material is from 5 to 95% (e.g., 20 to 90% and 40 to 85%) by weight of the capsule. The amount of the capsule wall is from 0.5% to 25% (e.g., 1.5 to 15% and 2.5 to 10%) also by weight of the capsule. In other embodiments, the amount of the encapsulated active material is from 15% to 99.5% (e.g., 50 to 98% and 30 to 95%) by weight of the capsule, and the amount of the capsule wall is from 0.5% to 85% (e.g., 2 to 50% and 5 to 70%) by weight of the capsule. Calculated by weight of the capsule composition, the amount of the encapsulated active material (e.g., a fragrance or flavor) can be 0.5% to 80%, preferably 5% to 60%, and more preferably 20% to 50%.

When the active material is a fragrance, in some embodiments, a fragrance having a high weighted C log P is encapsulated in the capsules of this invention, e.g., 3 to 8. In other embodiment, a fragrance having a low weighted C log P is used, e.g., 0.5 to 3. For instance, the ingredients having a C log P value 2 and 7 (e.g., 2 and 6, and 2 and 5) are 25% or greater (e.g., 50% or greater and 90% or greater) by the weight of the fragrance. Those skilled in the art will appreciate that many fragrances can be created employing various solvents and fragrance ingredients. The use of a wide range C log P fragrance ingredients will result in fragrances that are suitable for encapsulation. These fragrances are generally water-insoluble, to be delivered through the capsule composition of this invention onto consumer products in different stages such as damp and dry fabric. Without encapsulation, the free fragrances would normally have evaporated or dissolved in water during use, e.g., wash.

High C log P materials have excellent encapsulation properties they are generally well delivered from a regular (non-encapsulated) fragrance in a consumer product. Such fragrance chemicals would generally need encapsulation for overall fragrance character purposes, very long-lasting fragrance delivery, or overcoming incompatibility with the consumer product, e.g., fragrance materials that would otherwise be instable, cause thickening or discoloration of the product or otherwise negatively affect desired consumer product properties.

The active material to be encapsulated can be dispersed in aqueous solutions in the presence of a dispersant containing a fully hydrolyzed polyvinyl alcohol and a water-dispersible polymer, and in the absence/presence of other adjunct materials described below prior to formation of capsules.

### Adjunct Materials

In addition to the active materials, the present invention also contemplates the incorporation of adjunctive materials including solvent, emollients, and core modifier materials in the core encapsulated by the capsule wall. Other adjunct materials are solubility modifiers, density modifiers, stabilizers, viscosity modifiers, pH modifiers, or any combination thereof. These modifiers can be present in the wall or core of the capsules, or outside the capsules in the composition. Preferably, they are in the core as a core modifier.

The one or more adjunct material may be added in the amount of from 0.01% to 25% (e.g., from 0.5% to 10%) by weight of the capsule.

(i) Solvent. Preferable solvent materials are hydrophobic and miscible with the active materials. Solvents increase the compatibility of various active materials, increase the overall hydrophobicity of the mixture containing the active materials, influence the vapor pressure, or serve to structure the mixture. Suitable solvents are those having reasonable affinity for the active materials and a C log P greater than 2.5, preferably greater than 3.5 and more preferably greater than 5.5. In some embodiments, the solvent is combined with the active materials that have C log P values as set forth above. It should be noted that selecting a solvent and active material with high affinity for each other will result in improvement in stability. Exemplary solvents are triglyceride oil, mono and diglycerides, mineral oil, silicone oil, diethyl phthalate, polyalpha olefins, castor oil, isopropyl myristate, mono-, di- and tri-esters and mixtures thereof, fatty acids, and glycerine. The fatty acid chain can range from C₄-C₂₆ and can have any level of unsaturation. For instance, one of the following solvents can be used: capric/caprylic triglyceride known as NEOBEE M5 (Stepan Corporation); the CAPMUL series by Abitec Corporation (e.g., CAPMUL MCM); isopropyl myristate; fatty acid esters of polyglycerol oligomers, e.g., R²CO—[OCH₂—CH(OCOR¹)—CH²O—]ₙ, where R¹ and R² can be H or C₄-C₂₆ aliphatic chains, or mixtures thereof, and n ranges between 2 and 50, preferably 2 and 30; nonionic fatty alcohol alkoxylates like the NEODOL surfactants by BASF; the dobanol surfactants by Shell Corporation or the BIO-SOFT surfactants by Stepan, wherein the alkoxy group is ethoxy, propoxy, butoxy, or mixtures thereof and said surfactants can be end-capped with methyl groups in order to increase their hydrophobicity; di- and tri-fatty acid chain containing nonionic, anionic and cationic surfactants, and mixtures thereof; fatty acid esters of polyethylene glycol, polypropylene glycol, and polybutylene glycol, or mixtures thereof; polyalphaolefins such as the EXXONMOBIL PURESYM PAO line; esters such as the EXXONMOBIL PURESYN esters; mineral oil; silicone oils such polydimethyl siloxane and polydimethylcyclosiloxane; diethyl phthalate; di-octyl adipate and di-isodecyl adipate. In certain embodiments, ester oils have at least one ester group in the molecule. One type of common ester oil useful in the present invention are the fatty acid mono and polyesters such as cetyl octanoate, octyl isonanoanate, myristyl lactate, cetyl lactate, isopropyl myristate, myristyl myristate, isopropyl palmitate, isopropyl adipate, butyl stearate, decyl oleate, cholesterol isostearate, glycerol monostearate, glycerol distearate, glycerol tristearate, alkyl lactate, alkyl citrate and alkyl tartrate; sucrose ester and polyesters, sorbitol ester, and the like. A second type of useful ester oil is predominantly composed of triglycerides and modified triglycerides. These include vegetable oils such as jojoba, soybean, canola, sunflower, safflower, rice bran, avocado, almond, olive, sesame, persic, castor, coconut, and mink oils. Synthetic triglycerides can also be employed provided they are liquid at room temperature. Modified triglycerides include materials such as ethoxylated and maleated triglyceride derivatives provided they are liquids. Proprietary ester blends such as those sold by FINETEX as FINSOLV are also suitable, as is ethylhexanoic acid glyceride. A third type of ester oil is liquid polyester formed from the reaction of a dicarboxylic acid and a diol. Examples of polyesters suitable for the present invention are the polyesters marketed by EXXONMOBIL under the trade name PURESYN ESTER.

While the core can be free of the solvent, it is preferable that the level of solvent is 80 wt % or less, preferably 50 wt % or less (e.g., 0-20 wt %) by weight of the core.

(ii) Triglycerides and modified triglycerides as emollients. These include vegetable oils such as jojoba, soybean, canola, sunflower, safflower, rice bran, avocado, almond, olive, sesame, persic, castor, coconut, and mink oils.

(iii) Ester oils have at least one ester group in the molecule. One type of common ester oil useful in the present invention are the fatty acid mono and polyesters such as cetyl octanoate, octyl isonanoanate, myristyl lactate, cetyl lactate, isopropyl myristate, myristyl myristate, isopropyl palmitate, isopropyl adipate, butyl stearate, decyl oleate, cholesterol isostearate, glycerol monostearate, glycerol distearate, glycerol tristearate, alkyl lactate, alkyl citrate and alkyl tartrate.

(iv) Ester oil as a liquid polyester formed from the reaction of a dicarboxylic acid and a diol. Examples of polyesters suitable for the present invention are the polyesters marketed by ExxonMobil under the trade name PURESYN ESTER®, hydrophobic plant extracts.

(v) Silicones include, for example, linear and cyclic polydimethylsiloxanes, amino-modified, alkyl, aryl, and alkylaryl silicone oil.

(vi) Low/non-volatile hydrocarbons

(vii) Solid materials. Nanoscale solid particulate materials such as those disclosed in U.S. Pat. No. 7,833,960 may also be incorporated into the core and may be selected from, but not limited to, metal or metallic particles, metal alloys, polymer particles, wax particles, inorganic particulates, minerals and clay particles.

The metal particles can be selected from a non-limiting list of main group elements, transition metal and post-transition metal elements including aluminum (Al), silica (Si), Titanium (Ti), chromium (Cr), manganese (Mn), iron (Fe), nickel (Ni), cobalt (Co), copper (Cu), gold (Au), silver (Ag), platinum (Pt) and palladium (Pd).

Polymer particles of any chemical composition and nature are suitable for the present invention as long as their physical dimension falls into the prescribed region and a liquid core is generated. The polymer particles can be selected from a nonlimiting list of polymers and co-copolymer based on polystyrene, polyvinyl acetate, polylactides, polyglycolides, ethylene maleic anhydride copolymer, polyethylene, polypropylene, polyamide, polyimide, polycarbonate, polyester, polyurethane, polyurea, cellulose and cellulose, and combinations and mixture of such polymers.

The inorganic particulate can be selected from a non-limiting list including silica, titanium dioxide (TiO2), zinc oxide (ZnO), Fe2O3, and other metal oxides such as but not limited to NiO, Al2O3, SnO, SnO2, CeO2, ZnO, CdO, RuO2, FeO, CuO, AgO, MnO2, as well as other transition metal oxides.

Examples of nanoscaled material include AEROSIL R812, which has a particle size of less than 25 nm according to the specification from the manufacture, Degussa Corp. Other suitable materials from Degussa include, but not limited to, AEROSIL R972, AEROSIL R974, AEROSIL R104, AEROSIL R106, AEROSIL R202, AEROSIL R805, AEROSIL R812, AEROSIL R812S, AEROSIL R816, AEROSIL R7200, AEROSIL R9200, and AEROXIDE TiO2 P25, AEROXIDE T805, AEROXIDE LE1, AEROXIDE LE2, AEROXIDE TiO2 NKT 90, AEROXIDE Alu C805, titanium dioxide PF2, SIPERNAT D110, SIPERNAT D-380. The hydrophobic materials from Deguassa Corp. such as including AEROSILE R812 and R972 are especially preferred.

Nanoscaled materials such as UVINUL TiO₂ and Z-COTE HP1 manufactured by BASF can also be used as well as and TI-PURE titanium dioxide, TI-PURE R-700, and TI-SELECT. Additional suitable materials include TS-6200 from Dupont and ZEROFREE 516, HUBERDERM 2000 and HUBERDERM 1000 from the J.M. Huber Corporation, Havre De Grace, Md. Silica products such as SYLOID 63, 244, 72, 63FP 244FP, 72FP, SYLOX 15, 2 and Zeolites such as SYLOSIV A3, SYLOSIV A4 and SYLOSIV K300 from Grace Davison can also be used.

(viii) Polymeric core modifiers. Polymeric core modifiers are also contemplated. It has been found that the addition of hydrophobic polymers to the core can also improve stability by slowing diffusion of the fragrance from the core. The level of polymer is normally less than 80% of the core by weight, preferably less than 50%, and most preferably less than 20%. The basic requirement for the polymer is that it be miscible or compatible with the other components of the core, namely the fragrance and other solvent. Preferably, the polymer also thickens or gels the core, thus further reducing diffusion. Polymeric core modifiers include copolymers of ethylene; copolymers of ethylene and vinyl acetate (ELVAX polymers by DOW Corporation); copolymers of ethylene and vinyl alcohol (EVAL polymers by Kuraray); ethylene/acrylic elastomers such as VALNAC polymers by Dupont; polyvinyl polymers, such as polyvinyl acetate; alkyl-substituted cellulose, such as ethyl cellulose (ETHOCEL made by DOW Corporation) and hydroxypropyl celluloses (KLUCEL polymers by Hercules); cellulose acetate butyrate available from Eastman Chemical; polyacrylates (e.g., AMPHOMER, DEMACRYL LT and DERMACRYL 79, made by National Starch and Chemical Company, the AMERHOLD polymers by Amerchol Corporation, and ACUDYNE 258 by ISP Corporation); copolymers of acrylic or methacrylic acid and fatty esters of acrylic or methacrylic acid such as INTELIMER POLYMERS made by Landec Corporation (see also U.S. Pat. Nos. 4,830,855, 5,665,822, 5,783,302, 6,255,367 and 6,492,462); polypropylene oxide; polybutylene oxide of poly(tetrahydrofuran); polyethylene terephthalate; polyurethanes (DYNAM X by National Starch); alkyl esters of poly(methyl vinyl ether); maleic anhydride copolymers, such as the GANTREZ copolymers and OMNIREZ 2000 by ISP Corporation; carboxylic acid esters of polyamines, e.g., ester-terminated polyamides (ETPA) made by Arizona Chemical Company; polyvinyl pyrrolidone (LUVISKOL series of BASF); block copolymers of ethylene oxide, propylene oxide and/or butylenes oxide including, e.g., PLURONIC and SYNPERONIC polymers/dispersants by BASF. Another class of polymers include polyethylene oxide-co-propyleneoxide-co-butylene oxide polymers of any ethylene oxide/propylene oxide/butylene oxide ratio with cationic groups resulting in a net theoretical positive charge or equal to zero (amphoteric). The general structure is:

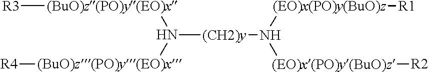

where R1, R2, R3, and R4 are independently H or any alkyl or fatty alkyl chain group. Examples of such polymers are the commercially known as TETRONICS by BASF Corporation.

(ix) Sacrificial core ingredients. These ingredients can also be included in the core and are designed to be lost during or after manufacture and include, but are not limited to, highly water soluble or volatile materials.

(x) Solubility modifiers. Nonlimiting examples of a solubility modifier include surfactants (e.g., SLS and Tween 80), acidic compounds (e.g., mineral acids such as sulfuric acid, hydrochloric acid, nitric acid, and phosphoric acid, and carboxylic acids such as acetic acid, citric acid, gluconic acid, glucoheptonic acid, and lactic acid), basic compounds (e.g., ammonia, alkali metal and alkaline earth metal hydroxides, primary, secondary, or tertiary amines, and primary, secondary, or tertiary alkanolamines), ethyl alcohol, glycerol, glucose, galactose, inositol, mannitol, glactitol, adonitol, arabitol, and amino acids.

(xi) Density modifiers. The density of the capsule slurry and/or the oil core can be adjusted so that the capsule composition has a substantially uniform distribution of the capsules using known density modifiers or technologies such as those described in Patent Application Publications WO 2000/059616, EP 1 502 646, and EP 2 204 155. Suitable density modifiers include hydrophobic materials and materials having a desired molecular weight (e.g., higher than about 12,000), such as silicone oils, petrolatums, vegetable oils, especially sunflower oil and rapeseed oil, and hydrophobic solvents having a desired density (e.g., less than about 1,000 Kg/m3 at 25° C., such as limonene and octane.

(xii) Stabilizers. In some embodiments, a stabilizer (e.g., a colloidal stabilizer) is added to a capsule composition to stabilize the emulsion and/or capsule slurry. Examples of colloidal stabilizers are polyvinyl alcohol, cellulose derivatives such hydroxyethyl cellulose, polyethylene oxide, copolymers of polyethylene oxide and polyethylene or polypropylene oxide, or copolymers of acrylamide and acrylic acid. In other embodiments, a stabilizing agent (i.e., a stabilizer) is added to the capsule composition to improve the stability of the composition for an extended period of storage. When one of these compositions is added to a consumer product such as a liquid fabric softener/freshener and liquid detergent, the capsule composition will also improve the viscosity stability of the consumer product, thus extend the shelf life of the product.

Useful stabilizing agents include multi-functional amines, amino acids/peptides, mono-functional amines, polymers, and a polymeric mixture. These stabilizing agents are in presence in the compositions as free compounds, which are not covalently attached to the capsule walls, being part of the capsule walls, or encapsulated in capsules.

Multi-functional amines are those having at least an amine group (primary, secondary, or tertiary) and one or more other functional groups such as an amine and hydroxyl group. Exemplary multi-functional amines include hexamethylenediamine, hexaethylenediamine, ethylenediamine, 1,3-diaminopropane, 1,4-diamino-butane, diethylenetriamine, pentaethylenehexamine, bis(3-aminopropyl)amine, bis(hexanethylene)triamine, tris(2-aminoethyl)amine, triethylene-tetramine, N,N′-bis(3-aminopropyl)-1,3-propanediamine, tetraethylenepentamine, amino-2-methyl-1-propanol branched polyethylenimine, chitosan, 1,3-diamino-guanidine, 1,1-dimethylbiguanide, and guanidine. Suitable amino acids/peptides include arginine, lysine, histidine, ornithine, nisin, and gelatin. Suitable stabilizing polymers include polyvinylpyrrolidone, polyvinylpyridine-N-oxide, and polyvinyl imidazolinium. These polymers sometimes are used in combination with a second polymer (e.g., a block copolymer) such that the second polymer.

Monofunational amines have a single amine group. Examples include C1-C20 primary, secondary, or tertiary amines, each of which typically has a molecular weight of 30 to 800 Daltons (e.g., 31 to 500 Daltons and 31 to 300 Daltons). They can be linear, branched, cyclic, acyclic, saturated, unsaturated, aliphatic, and/or aromatic. Nonlimiting examples are methylamine, dimethylamine, trimethylamine, ethylamine, diethylamine, triethylamine, propylamine, isopropylamine, butylamine, dodecylamine, tetradecylamine, aniline, 4-methylaniline, 2-nitroaniline, diphenyl amine, pyrrolidone, piperidine, and morpholine.

The stabilizing agent in the capsule composition can be present in an amount effective to stabilize the composition and/or the final consumer product containing the composition. This amount can be 1 ppm or greater (e.g., 20 ppm or greater, 20 ppm to 20%, 50 ppm to 10%, 50 ppm to 2%, 50 ppm to 1%, 50 to 2000 ppm, and 50 to 1000 ppm). Its concentration in a consumer product can be 20 ppm to 2% (e.g., 50 ppm to 2%, 50 ppm to 1%, 50 to 2000 ppm, and 50 to 1000 ppm).

(xiii) Viscosity control agents. Viscosity control agents (e.g., suspending agents), which may be polymeric or colloidal (e.g., modified cellulose polymers such as methylcellulose, hydoxyethylcellulose, hydrophobically modified hydroxyethylcellulose, and cross-linked acrylate polymers such as Carbomer, hydrophobically modified polyethers) can be included in the capsule composition, in the capsule core or wall, or in the capsule slurry outside the capsules. Optionally, silicas, either hydrophobic or hydrophilic, can be included at a concentration from 0.01 to 20%, more preferable from 0.5 to 5%, by the weight of the capsule composition. Examples of hydrophobic silicas include silanols, surfaces of which are treated with halogen silanes, alkoxysilanes, silazanes, and siloxanes, such as SIPERNAT D17, AEROSIL R972 and R974 available from Degussa. Exemplary hydrophilic silicas are AEROSIL 200, SIPERNAT 22S, SIPERNAT 505 (available from Degussa), and SYLOID 244 (available from Grace Davison).

(xiv) Humectants. One or more humectants are optionally included to hold water in the capsule composition for a long period of time. Examples include glycerin, propylene glycol, alkyl phosphate esters, quaternary amines, inorganic salts (e.g., potassium polymetaphosphate, sodium chloride, etc.), polyethylene glycols, and the like.

Further suitable humectants, as well as viscosity control/suspending agents, are disclosed in U.S. Pat. Nos. 4,428,869, 4,464,271, 4,446,032, and 6,930,078. Details of hydrophobic silicas as a functional delivery vehicle of active materials other than a free flow/anticaking agent are disclosed in U.S. Pat. Nos. 5,500,223 and 6,608,017.

(xv) pH modifiers. In some embodiments, one or more pH modifiers are included in the capsule composition to adjust the pH value of the capsule slurry and/or the capsule cores. The pH modifiers can also assist in the formation of capsule walls by changing the reaction rate of the crosslinking reactions that form the capsule walls. Exemplary pH modifiers include metal hydroxides (e.g., LiOH, NaOH, KOH, and Mg(OH)2), metal carbonates and bicarbonates (CsCO3 Li2CO3, K2CO3, NaHCO3, and CaCO3), metal phosphates/hydrogen phosphates/dihydrogen phosphates, metal sulfates, ammonia, mineral acids (HCl, H2SO4, H3PO4, and HNO3), carboxylic acids (e.g., acetic acid, citric acid, lactic acid, benzoic acid, and sulfonic acids), and amino acids.

(xvi) Formaldehyde scavengers are capable of binding free formaldehyde. When the capsules are for use in aqueous media, formaldehyde scavengers such as sodium sulfite, melamine, glycine, and carbohydrazine are suitable. When the capsules are aimed to be used in products having low pH, e.g., fabric care conditioners, formaldehyde scavengers are preferably selected from beta diketones, such as beta-ketoesters, or from 1,3-diols, such as propylene glycol. Preferred beta-ketoesters include alkyl-malonates, alkyl aceto acetates and polyvinyl alcohol aceto acetates. See US 2014/0044761.

### Deposition Aids

A capsule deposition aid from 0.01 to 25%, more preferably from 5 to 20% can be included by weight of the capsule. The capsule deposition aid can be added during the preparation of the capsules or it can be added after the capsules have been made.

These deposition aids are used to aid in deposition of capsules to surfaces such as fabric, hair or skin. These include anionically, cationically, nonionically, or amphoteric water-soluble polymers. Those skilled in the art would appreciate that the charge of these polymers can be adjusted by changing the pH, depending on the product in which this technology is to be used. Any suitable method for coating the deposition aids onto the encapsulated fragrance materials can be used. The nature of suitable polymers for assisted capsule delivery to interfaces depends on the compatibility with the capsule wall chemistry since there has to be some association to the capsule wall. This association can be through physical interactions, such as hydrogen bonding, ionic interactions, hydrophobic interactions, electron transfer interactions or, alternatively, the polymer coating could be chemically (covalently) grafted to the capsule or particle surface. Chemical modification of the capsule or particle surface is another way to optimize anchoring of the polymer coating to capsule or particle surface. Furthermore, the capsule and the polymer need to be compatible with the chemistry (polarity, for instance) of the desired interface. Therefore, depending on which capsule chemistry and interface (e.g., cotton, polyester, hair, skin, wool), the polymer can be selected from one or more polymers with an overall zero (amphoteric: mixture of cationic and anionic functional groups) or net positive charge, based on the following polymer backbones: polysaccharides, polypeptides, polycarbonates, polyesters, polyolefinic (vinyl, acrylic, acrylamide, poly diene), polyester, polyether, polyurethane, polyoxazoline, polyamine, silicone, polyphosphazine, olyaromatic, poly heterocyclic, or polyionene, with molecular weight (MW) ranging from about 1,000 to about 1000,000,000, preferably from about 5,000 to about 10,000,000. As used herein, molecular weight is provided as weight average molecular weight.

Particular examples of cationic polymers that can be used to coat the polyurea or polyurethane capsule include, e.g., polysaccharides such as guar, alginates, starch, xanthan, chitosan, cellulose, dextrans, arabic gum, carrageenan, and hyaluronates. These polysaccharides can be employed with cationic modification and alkoxy-cationic modifications such as cationic hydroxyethyl or cationic hydroxypropyl. For example, cationic reagents of choice are 3-chloro-2-hydroxypropyl trimethylammonium chloride or its epoxy version. Another example is graft-copolymers of polyDADMAC on cellulose. Alternatively, polysaccharides can be employed with aldehyde, carboxyl, succinate, acetate, alkyl, amide, sulfonate, ethoxy, propoxy, butoxy, and combinations of these functionalities; or any hydrophobic modification (compared to the polarity of the polysaccharide backbone). The above modifications can be in any ratio and the degree of functionalization can be up to complete substitution of all functionalizable groups, as long as the theoretical net charge of the polymer is zero (mixture of cationic and anionic functional groups) or preferably positive. Furthermore, up to 5 different types of functional groups may be attached to the polysaccharides. Also, polymer graft chains may be differently modified to the backbone. The counterions can be any halide ion or organic counter ion. See U.S. Pat. Nos. 6,297,203 and 6,200,554.

Another source of cationic polymers contain protonatable amine groups so that the overall net charge is zero (amphoteric: mixture of cationic and anionic functional groups) or positive. The pH during use will determine the overall net charge of the polymer. Examples include silk protein, zein, gelatin, keratin, collagen and any polypeptide, such as polylysine.

Further cationic polymers include polyvinyl polymers with up to 5 different types of monomers can be used. The monomers of such polymer have the generic formula:

—C(R₂)(R₁)—CR₂R₃—

wherein, R₁ is H, C₁-C₂₅ alkane, C₁-C₂₅ alkene (in which the number of double bonds ranges from 1-5), C₁-C₂₅ alkoxylated fatty alcohol, or a liquid crystalline moiety that can provide the polymer with thermotropic liquid crystalline properties;

R₂ is H or CH₃; and

R₃ is —Cl, —NH₂ (i.e., polyvinyl amine or its copolymers with N-vinyl formamide.

Such polyvinyl polymers are sold under the name LUPAMIN 9095 by BASF Corporation. Further suitable cationic polymers containing hydroxylalkylvinylamine units, as disclosed in U.S. Pat. No. 6,057,404.

Another class of materials are polyacrylates with up to 5 different types of monomers. Monomers of polyacrylates have the generic formula:

—CH(R₁)—C(R₂)(CO—R₃—R₄)—

wherein, R₁ is H, C₁-C₂₅ alkane, C₁-C₂₅ alkene (in which the number of double bonds ranges from 1-5), C₁-C₂₅ alkoxylated fatty alcohol, or a liquid crystalline moiety that can provide the polymer with thermotropic liquid crystalline properties;

R₂ is H or CH₃;

R₃ is a C₁-C₂₅ alkyl alcohol or an alkylene oxide with any number of double bonds, or R₃ may be absent such that the C═O bond is (via the C-atom) directly connected to R₄; and

R₄ is —NH₂, —NHR₁, —NR₁R₂, —NR₁R₂R₆ (where R₆═R₁, R₂, or —CH₂—COOH or its salt), —NH—C(O)—, sulfobetaine, betaine, polyethylene oxide, poly(ethyleneoxide/propylene oxide/butylene oxide) grafts with any end group, H, OH, styrene sulfonate, pyridine, quaternized pyridine, alkyl-substituted pyrrolidone or pyridine, pyridine-N-oxide, imidazolinium halide, imidazolium halide, imidazol, piperidine, —OR₁, —OH, —COOH alkali salt, sulfonate, ethoxy sulphate, pyrrolidone, caprolactam, phenyl-R₄ or naphthalene-R₅, where R₄ and R₅ are R₁, R₂, R₃, sulfonic acid or its alkali salt or organic counter ion. Also, glyoxylated cationic polyacrylamides can be used. Typical polymers of choice are those containing the cationic monomer dimethylaminoethyl methacrylate (DMAEMA) or methacrylamidopropyl trimethyl ammonium chloride (MAPTAC). DMAEMA can be found in GAFQUAT and GAFFIX VC-713 polymers from ISP. MAPTAC can be found in BASF's LUVIQUAT PQ11 PN and ISP's GAFQUAT HS100.

Another group of polymers that can be used are those that contain cationic groups in the main chain or backbone. Included in this group are:

i) polyalkylene imines such as polyethylene imine, commercially available as LUPASOL from BASF. Any molecular weight and any degree of crosslinking of this polymer can be used in the present invention;

ii) ionenes as disclosed in U.S. Pat. No. 4,395,541 and U.S. Pat. No. 4,597,962;

iii) adipic acid/dimethyl amino hydroxypropyl diethylene triamine copolymers, such as CARTARETIN F-4 and F-23, commercially available from Sandoz;

iv) polymers of the general formula: —[N(CH₃)₂—(CH₂)ₓ—NH—(CO)—NH—(CH₂)_{y}—N(CH₃)₂)—(CH₂)_{z}—O—(—CH₂)ₚ]ₙ—, with x, y, z, p=1-12, and n according to the molecular weight requirements. Examples are Polyquaternium-2 (MIRAPOL A-15), Polyquaternium-17 (MIRAPOL AD-1), and Polyquaternium-18 (MIRAPOL AZ-1). Other polymers include cationic polysiloxanes and cationic polysiloxanes with carbon-based grafts with a net theoretical positive charge or equal to zero (mixture of cationic and anionic functional groups). This includes cationic end-group functionalized silicones (i.e., Polyquaternium-80). Silicones with general structure: —Si(R₁)(R₂)—O—]ₓ—[Si(R₃)(R₂)—O—]_{y}— where R₁ is any alkane from C₁-C₂₅ or H with number of double bonds from 0-5, aromatic moieties, polysiloxane grafts, or mixtures thereof. R₁ can also be a liquid crystalline moiety that can provide the polymer with thermotropic liquid crystalline properties. R₂ can be H or CH₃; and R₃ can be —R₁-R₄, where R₄ can be —NH₂, —NHR₁, —NR₁R₂, —NR₁R₂R₆ (where R₆═R₁, R₂, or —CH₂—COOH or its salt), —NH—C(O)—, —COOH, —COO— alkali salt, any C₁-C₂₅ alcohol, —C(O)—NH₂ (amide), —C(O)—N(R₂)(R₂′)(R₂″), sulfobetaine, betaine, polyethylene oxide, poly(ethyleneoxide/propylene oxide/butylene oxide) grafts with any end group, H, —OH, styrene sulfonate, pyridine, quaternized pyridine, alkyl-substituted pyrrolidone or pyridine, pyridine-N-oxide, imidazolinium halide, imidazolium halide, imidazol, piperidine, pyrrolidone, caprolactam, sulfonate, ethoxysulphate phenyl-R₅ or naphthalene-R₆ where R₅ and R₆ are R₁, R₂, R₃, sulfonic acid or its alkali salt or organic counter ion. R₃ can also be —(CH₂)ₓ—O—CH₂—CH(OH)—CH₂—N(CH₃)₂—CH₂—COOH and its salts. Any mixture of these R₃ groups can be selected. X and y can be varied as long as the theoretical net charge of the polymer is zero (amphoteric) or positive. In addition, polysiloxanes containing up to 5 different types of monomeric units may be used. Examples of suitable polysiloxanes are found in U.S. Pat. Nos. 4,395,541 4,597,962 and 6,200,554. Another group of polymers that can be used to improve capsule/particle deposition are phospholipids that are modified with cationic polysiloxanes. Examples of these polymers are found in U.S. Pat. No. 5,849,313, WO Patent Application 95/18096A1 and European Patent No. 0737183B1.

Furthermore, copolymers of silicones and polysaccharides and proteins can be used (e.g., those commercially available as CRODASONE brand products).

Another class of polymers includes polyethylene oxide-co-propyleneoxide-co-butylene oxide polymers of any ethylene oxide/propylene oxide/butylene oxide ratio with cationic groups resulting in a net theoretical positive charge or equal to zero (amphoteric). Examples of such polymers are the commercially available TETRONIC brand polymers.

Suitable polyheterocyclic (the different molecules appearing in the backbone) polymers include the piperazine-alkylene main chain copolymers disclosed by Kashiki and Suzuki (1986) Ind. Eng. Chem. Fundam. 25:120-125.

Table 1 below shows polyquaternium polymers that can be used as deposition aids in this invention.

TABLE 1
Deposition Aids -- Cationic Polyquaternium Polymers

| Polyquaternium | Description | Commercial Products |
|---|---|---|
| 1 | Ethanol, 2,2′,2″-nitrilotris-, polymer with | Polyquad (Alcon) |
| | 1,4-dichloro-2-butene and N,N,N′,N′- | |
| | tetramethyl-2-butene-1,4-diamine | |
| 2 | Poly[bis(2-chloroethyl) ether-alt-1,3-bis[3- | Mirapol A-15 |
| | (dimethylamino)propyl]urea] | |
| 4 | Hydroxyethyl cellulose dimethyl | Celquat L-200, H-100, L-200 |
| | diallylammonium chloride copolymer; | |
| | Diallyldimethylammonium chloride- | |
| | hydroxyethyl cellulose copolymer | |
| 5 | Copolymer of acrylamide and quaternized | Merquat 5, RETEN (Hercules) |
| | dimethylammoniumethyl methacrylate | |
| 6 | Poly(diallyldimethylammonium chloride) | Merquat 100, 106, Mirapol 100 |
| 7 | Copolymer of acrylamide and | Merquat 550, 550L, 550PR, S, |
| | diallyldimethylammonium chloride | 7SPR, 740, 2200, Mirapol 550, |
| | | Polyquart 770/NA, |
| | | Conditioneze 7 |
| 8 | Methyl and Stearyl Dimethylaminoethyl | |
| | Methacrylate Quaternized with Dimethyl | |
| | Sulfate | |
| 9 | Polydimethylaminoethyl Methacrylate | |
| | Quaternized with Methyl Bromide | |
| 10 | Quaternized hydroxyethyl cellulose | Merquat 10, Celquat SC-230M, |
| | | SC-240C, SC-140C, Ucare |
| | | Polymer |
| 11 | Copolymer of vinylpyrrolidone and | Luviquat PQ 11PN, Gafquat |
| | quaternized dimethylaminoethyl | 775N, 440, 734, 775 |
| | methacrylate | |
| 12 | 2-Propenoic Acid, 2-Methyl-, Decahydro- | |
| | 1,4-Dimethyl-7-(1-Methylethyl)-1- | |
| | Phenanthrenyl)Methyl Ester, Polymer with | |
| | 2-(Diethylamino)Ethyl 2-Methyl-2- | |
| | Propenoate and Ethyl 2-Methyl-2- | |
| | Propenoate, compd. with Dimethyl Sulfate | |
| 13 | 2-Propenoic Acid, 2-Methyl-, 2-(Diethyl- | |
| | amino)Ethyl Ester, Polymer with Ethyl 2- | |
| | Methyl-2-Propenoate and 9-Octadecenyl | |
| | 2-Methyl-2-Propenoate, compd. with | |
| | Dimethyl Sulfate | |
| 14 | Ethanaminium, N,N,N-Trimethyl-2-[(2- | |
| | Methyl-1-Oxo-2-Propenyl)Oxy]-, Methyl | |
| | Sulfate, Homopolymer | |
| 15 | Ethanaminium, N,N,N-Trimethyl-2-[(2- | Rohagit KF 720F (Rohm |
| | Methyl-1-Oxo-2-Propenyl)Oxy]-, Chloride, | GmbH) |
| | Polymer with 2-Propenamide | |
| 16 | Copolymer of vinylpyrrolidone and | Luviquat FC 370, HM 552, |
| | quaternized vinylimidazole | Style, FC 550, Excellence |
| 17 | Poly(Oxy-1,2-Ethanediyl (Dimethyl- | Mirapol AD |
| | iminio)-1,3-Propanediylimino(l,6-Dioxo- | |
| | l,6-Hexanediyl)Imino-1,3-Propanediyl- | |
| | (Dimethyliminio)-1,2-Ethanediyl | |
| | Dichloride | |
| 18 | Poly[oxy-1,2-ethanediyl(dimethyliminio)- | Luviquat 500 |
| | 1,3-propanediylimino-(1,6-dioxo-1,6- | |
| | heptanediyl)imino-1,3-propanediyl- | |
| | (dimethyliminio)-1,2-ethanediyl | |
| | dichloride] | |
| 19 | Ethenol, polymer with | Arlatone PQ-220 (ICI |
| | aminomethyloxirane | Americas) |
| 20 | Ethenyl octadecyl ether, polymer with | Arlatone PQ-225 |
| | aminomethyloxirane | |
| 22 | Copolymer of Acrylic Acid and | Merquat 280, 281, 280SD, 295 |
| | Diallyldimethylammonium Chloride | |
| 24 | Cellulose, 2-[2-Hydroxy-3-(Trimethyl- | Quatrisoft Polymer LM-200 |
| | ammonio)Propoxy]Ethyl Ether, Chloride | (Dow Chemical) |
| | (Similar to PQ-10) | |
| 27 | Hexanediamide, N,N′-bis(3-(Dimethyl- | |
| | amino)Propyl)-, Polymer with N,N′-bis(3- | |
| | Dimethylamino)Propyl Urea and 1,1′- | |
| | Oxybis(2-Chloroethane), Block | |
| 28 | Copolymer of vinylpyrrolidone and meth- | Gafquat HS-100, Conditioneze |
| | acrylamidopropyl trimethylammonium | NT-10 |
| 29 | Chitosan, 2,3-Dihydroxypropyl-2- | Quaternized Chitosan |
| | Hydroxy-3-(Trimethylammonio)Propyl | |
| | Ether, Chloride | |
| 30 | Ethanaminium, NCarboxymethyl)-N,N- | Mexomere PX (Chimex) |
| | Dimethyl-2-((2-Methyl-1-Oxo-2- | |
| | Propenyl)Oxy)-, Inner Salt, Polymer with | |
| | Methyl 2-Methyl-2-Propenoate | |
| 31 | 2-Propenenitrile, Homopolymer, | Hypan QT100 (Lipo) |
| | Hydrolyzed, Block, Reaction Products | |
| | with N,N-Dimethyl-1,3-Propanediamine, | |
| | Di-Et Sulfate-Quaternized | |
| 32 | Poly(acrylamide 2-methacryloxyethyl- | Cosmedia CTC (Cognis |
| | trimethyl ammonium chloride) | GmbH) - PQ-32 + other, |
| | | Salcare SC92 (Ciba Corp.) PQ- |
| | | 32 + other |
| 33 | Ethanaminium, N,N,N-Trimethyl-2-[1- | Lanoquat DES-50, Ultimer |
| | Oxo-2-Propenyl)Oxy]-, Chloride, Polymer | CG-200 (Nalco), Sepigel |
| | with 2-Propenamide | Quat33 (Seppic) - PQ-33 + |
| | | other |
| 34 | Poly(diethyliminio-1,3-propanediyldi- | Mexomere PAK (Chimex) |
| | methyliminio-1,3-propanediyl dibromide) | |
| 35 | Ethanaminium, N-carboxymethyl-N,N- | Plex 3074 L (Rohm GmbH) |
| | dimethyl-2-(2-methyl-1-oxo-2- | |
| | propenyloxy)-, inner salt, polymer with | |
| | N,N,N-trimethyl-2-(2-methyl-1-oxo-2- | |
| | propenyloxy)ethanaminium methyl sulfate | |
| 36 | 2-Propenoic Acid, 2-Methyl-, 2- | Plex 4739L (Rohm GmbH) |
| | (Dimethylamino)Ethyl Ester, Polymer with | |
| | Methyl 2-Methyl-2-Propenoate, compd. | |
| | with Dimethyl Sulfate | |
| 37 | N,N,N-Trimethyl-2-[(Methyl-1-Oxo-2- | Ultragel 300 (Cognis), |
| | Propenyl)Oxy]Ethanaminium Chloride, | Synthalen CN, CR, CU (3V |
| | Homopolymer | Group), Syntran PC 5320 |
| | | (Interpolymer) |
| 39 | 2-Propen-1-aminium, N,NDimethyl-N-2- | Merquat 3940, PLUS-3330, |
| | Propenyl-, Chloride, Polymer with 2- | PLUS-3331, 3331PR |
| | Propenamide and 2-Propenoic Acid | |
| 42 | Poly[oxyethylene(dimethyliminio)ethylene | Busan 1507 (Buckman Labs) |
| | (dimethylimino)ethylene dichloride] | |
| 43 | polymeric quaternary ammonium salt | Genamin PQ 43 (Clariant |
| | formed from acrylamide, | Functional Chemicals), |
| | acrylamidopropyltrimonium chloride, 2- | Bozequat 4000 (Clariant) |
| | amidopropylacrylamide sulfonate, and | |
| | DMAPA monomers | |
| 44 | Poly(2-oxopyrrolidin-1-ylethylene, 3- | Luviquat Ultracare, MS 370 |
| | methylimidazolium-1-ylethylene methyl | (BASF), Softenol PQ44 |
| | sulfate) | (Zdchimmer & Schwarz |
| | | Italianat S.p.A) |
| 45 | Glycine, N-methyl-N-[2-[(2-methyl-1-oxo- | Plex 3073L (Rohm GmbH) |
| | 2-propenyl)oxy] ethyl]-, polymer with 2- | |
| | (dimethylamino)ethyl 2-methyl-2- | |
| | propenoate, compound with dimethyl | |
| | sulfate | |
| 46 | 1H-Imidazolium, 1-Ethenyl-3-Methyl-, | Luviquat Hold |
| | Methyl Sulfate, Polymer with 1-Ethenyl- | |
| | hexahydro-2H-Azepin-2-one and 1- | |
| | Ethenyl-2-Pyrrolildinone | |
| 47 | 1-Propanaminium, N,N,NTrimethyl-3-((2- | Merquat 2001, 2001N |
| | Methyl-1-Oxo-2-Propenyl)Amino)-, | |
| | Chloride, Polymer with Methyl 2- | |
| | Propenoate and 2-Propenoic Acid | |
| 48 | Polymeric quaternary ammonium salt of | Plascize L-450 (Goo Chemical) |
| | formed from methacryloyl ethyl betaine, 2- | |
| | hydroxyethyl methacrylate and | |
| | methacryloyl ethyl trimethyl ammonium | |
| | chloride | |
| 49 | polymeric quaternary ammonium salt | Plascize L-440 |
| | formed by the reaction of methacryloyl | (Goo Chemical) |
| | ethyl betaine, PEG-9 methacrylate and | |
| | methacryloyl ethyl trimethyl ammonium | |
| | chloride | |
| 50 | Carboxylatoethyldimethylammonioethyl 2- | Plascize L-401 (Goo Chemical) |
| | methyl-2-propenoate homopolymer | |
| 51 | 3,5,8-Triox-4-Phosphaundec-10-en-1- | Lipidure PMB |
| | aminium, 4-Hydroxy-N,N,N,10- | (NOF) |
| | Tetramethyl-9-Oxo, Inner Salt, 4-Oxide, | |
| | Polymer with Butyl 2-Methyl-2- | |
| | Propenoate | |
| 53 | Acrylic Acid/Acrylamide/Methacryl- | Merquat 2003PR |
| | amidopropyltrimonium Chloride | |
| | Copolymer | |
| 54 | Aspartic acid, polymer with C6-18 | Quilty-Hy (Mitsui) |
| | alkylamine, 3-dimethylaminopropylamine | |
| | and sodium chloroacetate | |
| 55 | 1-Dodecanaminium, N,NDimethyl-N-[3- | Styreze W |
| | [(2-Methyl-1-Oxo-2-Propenyl)-Amino- | |
| | Propyl]-, Chloride, Polymer with N-[3- | |
| | (Dimethylamino)Propyl]-2-Methyl-2- | |
| | Propenamide and 1-Ethenyl-2- | |
| | Pyrrolidinone | |
| 56 | 5-Isocyanato-1-(isocyanatomethyl)-1,3,3- | Hairrol UC-4 (Sanyo |
| | trimethylcyclohexane, polymer with 1,3- | Chemical) |
| | butanediol and bis(2-hydroxyethyl)di- | |
| | methylammonium methyl sulfate | |
| 57 | 12-Hydroxy-9(Z)-octadecenamidopropyl- | Zenigloss Q (Zenitech) |
| | trimethylammonium chloride, polymers | |
| | with ricinus communis (castor) oil, | |
| | isooctdecanoic acid and butandioic acid | |
| 58 | 2-Propenoic Acid, Methyl Ester, Polymer | Lowenol Conditioner PWW |
| | with 2,2-Bis[(2-Propenyloxy)Methyl]-1- | (Lowenstein) -PQ-58 and |
| | Butanol and Diethenylbenzene, Reaction | Wheat Protein |
| | Products with N,NDimethyl-1,3-Propane- | |
| | diamine, Chloromethane-Quaternized | |
| 59 | Poly(20,25-dioxo-2,5,10,15,18-penta- | Crodasorb UV-HPP (Croda, |
| | methyl-10-(2-hydroxy-3-(3-(3-phenyl-2- | Inc.) - PQ-59 and Butylene |
| | propenamido)propyldimethylammonio)pro- | Glycol |
| | pyl)-10-azonia-1,4,7,13,16,19-hexaoxa- | |
| | pentacosanediyl) chloride | |
| 60 | 9-Octadecenoic Acid, 12-Hydroxy-, [(2- | Polylipid PPI-RC |
| | Hydroxyethyl)-Imino]Di-2,1-Ethanediyl | (Alzo/Bernel) - PQ-60 and |
| | Ester, Polymer with 5-Isocyanato-1- | Propylene Glycol |
| | (Isocyanatomethyl)-1,3,3-Trimethyl- | |
| | cyclohexane, Compd. with Diethyl Sulfate | |
| 61 | 2-Methyl-2-propenoyloxyethyl N,N,N- | Lipidure-S (NOF) |
| | trimethylammonioethyl phosphate inner | |
| | salt, polymer with octadecyl 2-methyl-2- | |
| | propenoate | |
| 62 | Polymeric quaternary ammonium salt of | Nanoaquasome (Amore |
| | butyl methacrylate, polyethylene glycol | Pacific/Kyung-do) |
| | methyl ether methacrylate, ethylene glycol | |
| | dimethacrylate and 2-methacryloylethyl | |
| | trimonium chloride with 2,2′-azobis(2- | |
| | methyl propionamidine)dihydrochloride | |
| 63 | polymeric quaternary ammonium salt | Finquat (Innospec), Octacare |
| | formed by acrylamide, acrylic acid and | PQ63 (Innospec Edison, NJ), |
| | ethyltrimonium chloride acrylate | OF-308 (WSP Chemical & |
| | | Technology) |
| 64 | 2-Methyl-2-propenoyloxyethyl N,N,N- | Lipidure-C (NOF) |
| | trimethylammonioethyl phosphate inner | |
| | salt, polymer with 2-hydroxy-3-(2-methyl- | |
| | 2-propenoyl)oxypropyltrimethyl- | |
| | ammonium chloride | |
| 65 | 2-Methyl-2-propenoyloxyethyl N,N,N- | Lipidure-A (NOF) |
| | trimethylammonioethyl phosphate inner | |
| | salt, polymer with butyl 2-methyl-2- | |
| | propenoate and sodium 2-methyl-2- | |
| | propenoate | |
| 66 | 5-Isocyanato-1-(isocyanatomethyl)-1,3,3- | WBR-2925C (Taisei) - PQ-66 |
| | trimethylcyclohexane, polymer with | and Methyl Pyrrolidone |
| | di(hydroxypolymethylene) benzene- | |
| | dicarboxylate and ethylbis(2-hydroxy- | |
| | ethyl)methylammonium ethyl sulfate | |
| 67 | 2-Hydroxyethyl cellulose ether, reaction | Softcat (Dow Chemical) |
| | products with N,N,N-trimethyl-N- | |
| | oxiranylmethylammonium chloride and N- | |
| | dodecyl-N,N-dimethyl-N- | |
| | oxiranylmethylammonium chloride | |
| 68 | 1-Ethenyl-2-pyrrolidinone, polymer with | Luviquat Supreme |
| | 1-ethenylimidazole and 1-ethenyl-3- | |
| | methylimidazolium methyl sulfate | |
| 69 | polymeric quaternary ammonium salt | Aquastyle 100, 300 (ISP) |
| | composed of vinyl caprolactam, | |
| | vinylpyrrolidone, dimethylaminopropyl | |
| | methacrylamide (DMAPA), and | |
| | methacryloylaminopropyl lauryldimonium | |
| | chloride | |
| 70 | polymeric quaternary ammonium salt | Lustreplex (Croda) |
| | consisting of an ethoxylated, propoxylated | |
| | stearyl amine condensed with adipic acid | |
| | and dilinoleic acid and quaternized with | |
| | dimethyl sulfate | |
| 71 | | ColaMoist 300P (Colonial |
| | | Chemical Inc) |
| 72 | polymeric quaternary ammonium salt of | Mirustyle CP (Croda) |
| | hydroxethylcellulose reacted with a coco- | |
| | alkyl dimethyl ammonium substituted | |
| | epoxide | |
| 73 | polymeric quaternary ammonium salt | Diaformer C-802, C-823 |
| | consisting of propyltrimonium chloride | (Mitsubishi Chem), Diasleek |
| | acrylamide, ethyltrimonium chloride | C-802, C-823 (Mitsubishi |
| | methacrylate and dimethylacrylamide | Chem) |
| | monomers; Propanaminium, N,N,N- | |
| | trimethyl-3-(2-propenamido)-, chloride, | |
| | polymer with N,N,N-trimethyl-2-(2- | |
| | methyl-2-propenoyloxy)ethanaminium | |
| | chloride and N,N-dimethyl-2-propenamide | |
| 74 | | Mirapol PB 20 (Rhodia) |
| | | Polycare Boost (Rhodia) |
| 75 | O-(2-Hydroxy-2-trimethylammonio- | Amylomer Cat 220EMU |
| | propyl)starch chloride, reaction products | (Grafe Chemie) |
| | with O-(3-dodecyldimethylammonio-2- | |
| | hydroxypropyl)starch chloride | |
| 76 | | Mirapol AT-1 (Rhodia) |
| 77 | Cocoglucoside Crosspolymer | Colonial Poly SugaQuat TM- |
| | Hydroxypropyltrimonium Chloride | 8610P (Colonial Chemical Inc) |
| 78 | Decylglucoside Crosspolymer | Colonial Poly SugaQuat L- |
| | Hydroxypropyl Laurdimonium Chloride | 1010P (Colonial Chemical Inc) |
| 79 | Decylglucoside Crosspolymer | Colonial Poly SugaQuat S- |
| | Hydroxypropyl Steardimonium Chloride | 1010P (Colonial Chemical Inc) |
| 80 | Laurylglucoside Crosspolymer | Colonial Poly SugaQuat L- |
| | Hydroxypropyl Laurdimonium Chloride | 1210P (Colonial Chemical Inc) |
| 81 | Laurylglucoside Crosspolymer | Colonial Poly SugaQuat S- |
| | Hydroxypropyl Steardimonium Chloride | 1210P (Colonial Chemical Inc) |
| 82 | Laurylglucoside Crosspolymer | Colonial Poly SugaQuat TM- |
| | Hydroxypropyltrimonium Chloride | 1218P (Colonial Chemical Inc) |
| 84 | polymeric quaternary ammonium salt of | Diasleek C-824 (Mitsubishi |
| | acrylamidopropyltrimethylammonium | Chemical) |
| | chloride, trimethylaminoethyl | |
| | methacrylate, dimethylacrylamide and | |
| | hydroxyethylmethacrylate | |
| 85 | polymeric quaternary ammonium salt of | Diasleek C-825 (Mitsubishi |
| | acrylamidopropyltrimethylammonium | Chemical) |
| | chloride, dimethylacrylamide and | |
| | hydroxyethylmethacrylate | |
| 86 | polymeric quaternary ammonium salt of | Luvigel Advanced (BASF) |
| | vinylpyrrolidone, 1-methyl-3- | |
| | vinylimidazoline chloride, vinylimidazole | |
| | and methacrylic acid | |
| 87 | polymeric quaternary ammonium salt of | Luviquat Sensation (BASF) |
| | vinylpyrrolidone, vinylimidazole and | |
| | diallyldimethyl ammonium chloride | |
| 88 | Poly(Dilinoleyldimonium | ColaQuat PDQ (Colonial |
| | hydroxypropyl)chlorides) | Chemical Inc) |
| 89 | polymeric quaternary ammonium salt | (BASF) |
| | prepared by the reaction of t-butyl acrylate, | |
| | vinyl pyrolidone, dimethylaminopropyl | |
| | methacrylamide, methacrylic acid and | |
| | ethyldimethyl[2-[(2-methyl-1- | |
| | oxoallyl)oxy]ammonium ethyl sulfate, | |
| | neutralized with orthophosphoric acid | |
| 90 | polymeric quaternary ammonium salt of | Hymoquat AK325R (Hymo |
| | acrylamide and hydroxyethylcellulose | Corporation) |
| | quaternized with diallyldimethyl | |
| | ammonium chloride | |
| 91 | polymeric quaternary ammonium salt of | Syntran 5500 (Interpolymer) - |
| | hydroxypropyl methacrylate and | PQ-91 and PA |
| | polyethylene glycol methacrylate | |
| | quaternized with ethyltrimonium chloride | |
| | methacrylate | |
| 92 | GLYCERYLAMIDOETHYL | Ceracute-G (NOF) |
| | METHACRYLATE/STEARYL | |
| | METHACRYLATE COPOLYMER | |
| 94 | polymeric quaternary ammonium salt | (Toho) |
| | consisting of acrylamide, dimethyl diallyl | |
| | ammonium chloride and | |
| | methacrylamidopropyltrimonium chloride | |
| | monomers | |
| 95 | copolymer of Zea Mays (Corn) Starch, | Polyquart Ecoclean (Cognis) |
| | Acrylic Acid and | |
| | acrylamidopropyltrimethylammonium | |
| | chloride monomers | |
| 98 | | (Cognis GmbH) |
| 101 | | Deposilk Q1 (Air Products) |

Other suitable deposition aids include those described in US 2013/0330292, US 2013/0337023, US 2014/0017278.

### Dosage Forms

The capsule composition of this invention can be a slurry or in a solid form for use in consumer products.

As a slurry, the capsule composition each contain a solvent (e.g., water) and the capsule at a level 0.1 to 80% (preferably 1 to 65% and more preferably 5 to 45%) by weight of the capsule composition.

In some embodiments, the capsule and its slurry prepared in accordance with the present invention is subsequently purified as described above.

The capsule slurry can also be spray dried to a solid form. In a spray drying process, a spray dry carrier is added to a capsule slurry to assist the removal of water from the slurry.

According to one embodiment, the spray dry carriers can be selected from the group consisting of carbohydrates such as chemically modified starches and/or hydrolyzed starches, gums such as gum arabic, proteins such as whey protein, cellulose derivatives, clays, synthetic water-soluble polymers and/or copolymers such as polyvinyl pyrrolidone, polyvinyl alcohol. The spray dry carriers may be present in an amount from 1 to 50%, more preferably from 5 to 20%.

Optionally, a free flow agent (anticaking agent) of silicas which may be hydrophobic (i.e. silanol surface treated with halogen silanes, alkoxysilanes, silazanes, siloxanes, etc. such as Sipemat D17, Aerosil R972 and R974 (available from Degussa), etc.) and/or hydrophilic such as Aerosil 200, Sipemat 22S, Sipemat 505, (available from Degussa), Syloid 244 (available from Grace Davison), may be present from about 0.01% to about 10%, more preferable from 0.5% to about 5%.

Humectants and viscosity control/suspending agents can also be added to facilitate spray drying. These agents are disclosed in U.S. Pat. Nos. 4,428,869, 4,464,271, 4,446,032, and 6,930,078. Details of hydrophobic silicas as a functional delivery vehicle of active materials other than a free flow/anticaking agent are disclosed in U.S. Pat. Nos. 5,500,223 and 6,608,017.

The spray drying inlet temperature is in the range of 150 to 240° C., preferably between 170 and 230° C., more preferably between 190 and 220° C.

As described herein, the spray-dried capsule composition is well suited for use in a variety of all dry (anhydrous) products: powder laundry detergent, fabric softener dryer sheets, household cleaning dry wipes, powder dish detergent, floor cleaning cloths, or any dry form of personal care products (e.g. shampoo powder, deodorant powder, foot powder, soap powder, baby powder), etc. Because of high fragrance and/or active agent concentration in the spray-dried products of the present invention, characteristics of the aforementioned consumer dry products will not be adversely affected by a small dosage of the spray-dried products.

The capsule composition can also be sprayed as a slurry onto a consumer product, e.g., a fabric care product. By way of illustration, a liquid capsule composition is sprayed onto a detergent powder during blending to make granules. See US 2011/0190191. In order to increase fragrance load, water-absorbing material, such as zeolite, can be added to the capsule composition.

Alternatively, granulates in a consumer product are prepared in a mechanical granulator in the presence of a granulation auxiliary such as non-acid water-soluble organic crystalline solids. See WO 2005/097962.

### Other Delivery Compositions

The capsule compositions of this invention can also contain one or more other delivery compositions such as polymer-assisted delivery compositions (see U.S. Pat. No. 8,187,580), fiber-assisted delivery compositions (US 2010/0305021), cyclodextrin host guest complexes (U.S. Pat. No. 6,287,603 and US 2002/0019369), pro-fragrances (WO 2000/072816 and EP 0 922 084), membrane delivery systems (U.S. Pat. No. 4,948,047), and any combination thereof.

The capsule composition can also contain one or more (e.g., two, three, four, five or six more) different capsules including different capsules of this invention and other capsules such as such as aminoplasts, hydrogel, sol-gel, coascervate capsules, polyurea/polyurethane capsules, and melamine formaldehyde capsules. More exemplary delivery systems that can be incorporated are coascervate capsules, cyclodextrin delivery systems, and pro-perfumes.

(1) Melt extruded flavor/fragrance. Polymer assisted delivery system include melt extruded flavor/fragrance utilizing high molecular weight carbohydrates, low molecular weight carbohydrates, or polymer.

(1.1) High molecular weight carbohydrate including starches and modified starches.

(1.2) Low molecular weight carbohydrates of a low molecular weight carbohydrate or polyol, wherein said low molecular weight carbohydrate or polyol is selected from the group consisting of glucose, sucrose, maltose, lactose, corn syrup solid, erythritol, lactitol, mannitol, sorbitol, maltitol, isomalt, xylitol, trehalose, hydrogenated corn syrup, hydrogenated glucose syrup, hydrogenated maltose syrup, hydrogenated lactose syrup, starch hydrolysate, and a mixture thereof, and wherein said glassy matrix has a glass transition temperature of greater than room temperature.

(1.3) Polymers (various polymers are useful in the practice of our invention. Specific examples of polymers useful in the practice of our invention are as follows: DYLAN™ of low density polyethylene (DYLAN™ is a trademark owned by the Atlantic Richfield Company of Los Angeles, Calif. DYLITE™ of expandable polystyrene compositions. DYLITE™ is a trademark of the Atlantic Richfield Company of Los Angeles, Calif. SUPER DYLAN™ of high density polyethylene. SUPER DYLAN™ a trademark of the Atlantic Richfield Company of Los Angeles, Calif.

Blended polyethylene and carbon black as specifically taught in U.S. Pat. No. 4,369,267 issued on Jan. 18, 1983, the specification for which is incorporated by reference herein.

Polystyrene as disclosed in U.S. Pat. No. 4,369,227 issued on Jan. 18, 1983, the specification for which is incorporated by reference herein. Polyene/alpha-olefin copolymers as exemplified and disclosed in U.S. Pat. No. 4,369,291, the specification for which is incorporated by reference herein. Poly-alpha-olefins as exemplified in Canadian Letters Pat. No. 1,137,069 issued on Dec. 7, 1982, the specification for which is incorporated by reference herein. Polymeric compositions as disclosed in Canadian Letters Pat. No. 1,137,068 issued on Dec. 7, 1982, the specification for which is incorporated by reference herein. Poly-alpha-olefins disclosed in Canadian Letters Pat. No. 1,137,067, the specification for which is incorporated by reference herein.

Polyolefins described in Canadian Letters Pat. No. 1,137,066, the specification for which is incorporated by reference herein. Polyethylene oxides as disclosed in Canadian Letters Pat. No. 1,137,065 issued on Dec. 7, 1982, the specification for which is incorporated by reference herein.

Olefin polymers and co-polymers as disclosed in Canadian Letters Pat. No. 1,139,737, the disclosure of which is incorporated by reference herein. Canadian Pat. No. 1,139,737 was issued on Jan. 18, 1983. Polyolefins disclosed in Canadian Letters Pat. No. 1,139,738, the specification for which is incorporated by reference herein. Canadian Pat. No. 1,139,738 was issued on Jan. 18, 1983. Chlorinated PVC as disclosed in Polymer 1982, 23 (7, Suppl.), 1051-6 abstracted at Chem. Abstracts 97:145570y, 1982.

Polyepsilon caprolactone co-polymers made by means of alcohol initiated polymerization as disclosed in J. Polym. Sci. Polym. Chem. Ed. 1982, 20(2), pages 319-26, abstracted at Chem. Abstracts, Volume 96: 123625x, 1982. Styrene acrylonitrile co-polymers as disclosed in Diss. Abstracts, Int. B, 1982, 42(8), 3346 and abstracted at Chem. Abstracts 96:143750n (1982). Co-polymers of epsilon caprolactone with 1,4-butane diol as disclosed at Kauch. Rezine, 1982, (2), 8-9, abstracted at Chem. Abstracts, volume 96:182506g (1982). Polyesters as disclosed in U.S. Pat. No. 4,326,010, the specification for which is incorporated by reference herein.

Chlorinated polyethylene as disclosed by Belorgey, et. al. J. Polym. Sci. Polym. Phys. Ed. 1982, 20(2), 191-203. Plasticized polyepsilon caprolactone co-polymers containing dimethyl phthalate plasticizers as set forth in Japanese Pat. No. J81/147844, abstracted at Chem. Abstracts, Volume 96:69984y (1982), the specification for which is incorporated by reference herein. Maleic anhydride modified adducts of polyepsilon caprolactone polyols and ethylenically unsaturated monomer as disclosed in U.S. Pat. No. 4,137,279 issued on Jan. 30, 1979, the specification for which is incorporated by reference herein. Polyurethane polymers having lactone backbones as disclosed in U.S. Pat. No. 4,156,067 issued on May 22, 1979, the disclosure of which is incorporated by reference herein. Polyurethane polyether resins wherein the resin is obtained by reacting a polyfunctional lactone with a long chain polyalkylene diol and a urethane precursor as disclosed in U.S. Pat. No. 4,355,550 issued on Mar. 10, 1981, the disclosure of which is incorporated by reference herein. Resins having polyurethane backbones as disclosed in U.S. Pat. No. 3,975,350 issued on Aug. 17, 1976, the disclosure of which is incorporated by reference herein.

(1.4) Suitable plasticizers include water; glycerol; propylene glycol; aqueous solutions of glycerol, propylene glycol, monosaccharides, and disaccharides; and invert and high fructose corn syrups.

(1.5) Emulsifier. surface-active agent, i.e. an emulsifier can be added to the dry blend, or preferably added to the liquid flavor mix which is ultimately injected into the metering zone of the extruder. These emulsifiers can be from the class of distilled monoglycerides, mono- and diglyceride blends, propyleneglycol monoglycerides, lecithin, modified lecithins, acetylated monoglycerides, lactylated monoglycerides, lactylated propyleneglycol monoglycerides, sorbitan esters, sorbitan-polyoxyethylene [20] monoglycerides, polyglycerol esters, DATEM's (diacetyltartarate esters of monoglycerides), succinylated esters of monoglycerides and polyoxyethylenepropylene copolymers and mixtures thereof. Most preferred surfactants are the sorbitan-polyoxyethylene [20] monoglycerides, lecithins, and polyglycerol esters.

(2) Spray Dry Encapsulation.

(2.1) The matrix is comprised of one or more of the following materials: sugars such as glucose, fructose, lactose, galactose, ribose, xylose, sucrose, maltose; polyols such as glycerin and propylene glycol; corn syrups, maltodextrin, fats, silicone dioxide, polyhydric alcohols, corn syrup solids, starches, modified starches, emulsifiers and food acids. The level of maltodextrin used in the matrix, comprises from about 25 to about 98 weight percent, preferably form about 35 to about 75 weight percent, the maltodextrin

(2.2) Core modifiers: flavors and fragrance may also be combined with a variety of solvents which serve to increase the compatibility of the various materials, increase the overall hydrophobicity of the blend, influence the vapor pressure of the materials, or serve to structure the blend. Solvents performing these functions are well known in the art and include mineral oils, triglyceride oils, silicone oils, fats, waxes, fatty alcohols, diisodecyl adipate, and diethyl phthalate among others.

(2.3) emulsifiers including monoglycerides of fatty acids, distilled succinylated monoglycerides of fatty acids, sorbitan fatty acid esters; distilled acetylated monoglycerides of fatty acids, monoglycerides of fatty acids.

(3) Coascervate Capsules.

(3.1) Proteins useful in coacervation processes include albumins, vegetable globulins and gelatines. The gelatine may be fish, pork, beef, and/or poultry gelatine, for example. According to a preferred embodiment, the protein is fish, beef or poultry gelatine. According to a more preferred embodiment, the protein is warm water fish gelatine.

(3.2) Typical non-protein polymers useful in complex coacervation methods include, in particular, negatively charged polymers. For example, they may be selected from gum arabic, xanthan, agar, alginate salts, cellulose derivatives, for example carboxymethyl cellulose, pectinate salts, carrageenan, polyacrylic and methacrylic acid, and/or mixtures thereof. Further suitable non-proteins can be derived from the literature, for example from to WO 2004/022221, page 4, lines 27-29

(3.3) A cross-linking agent is typically used to harden the coating layer. Suitable cross-linking agents include formaldehyde, acetaldehyde, glutaraldehyde, glyoxal, chrome alum, or transglutaminase. Preferably, transglutaminase is used at 10-100, preferably 30-60 activity units per gram of gelatine. This enzyme is well described and commercially obtainable.

(4) Cyclodextrin Delivery System

This technology approach uses a cyclic oligosaccharide or cyclodextrin to improve the delivery of perfume. Typically, a perfume and cyclodextrin (CD) complex is formed. Such complexes may be preformed, formed in-situ, or formed on or in the situs. See, e.g., WO 2013/109798 A2 and US 2011/0308556 A1.

(5) Pro-Perfume

(5.1) Michael Addition reaction products of a primary/secondary amine with an unsaturated ester, acid or nitrile perfume compound such those described in U.S. Pat. No. 6,858,575.

(5.2) Reaction product between a primary/secondary amine compound/polymer and a ketone or aldehyde perfume compound such as those described in WO 2001/051599 A1 and WO 2002/092746 A1

(5.3) other nonlimiting examples include aromatic or non-aromatic imines (Schiff bases), oxazolidines, beta-keto esters, orthoesters, compounds comprising one or more beta-oxy or beta-thio carbonyl moieties capable of releasing a perfume (e.g., an alpha, beta-unsaturated ketone, aldehyde or carboxylic ester). The typical trigger for perfume release is exposure to water; although other triggers may include enzymes, heat, light, pH change, autoxidation, a shift of equilibrium, change in concentration or ionic strength and others. Suitable pro-perfumes and methods of making same can be found in U.S. Pat. Nos. 8,912,350 B2, 7,018,978 B2; 6,987,084 B2; 6,956,013 B2; 6,861,402 B1; 6,544,945 B1; 6,093,691; 6,277,796 B1; 6,165,953; 6,316,397 B1; 6,437,150 B1; 6,479,682 B1; 6,096,918; 6,218,355 B1; 6,133,228; 6,147,037; 7,109,153 B2; 7,071,151 B2; 6,987,084 B2; 6,916,769 B2; 6,610,646 B2 and 5,958,870, as well as can be found in US 2005/0003980 A1 and US 2006/0223726 A1.

Any compound, polymer, or agent discussed above can be the compound, polymer, or agent itself as shown above, or its salt, precursor, hydrate, or solvate. A salt can be formed between an anion and a positively charged group on the compound, polymer, or agent. Suitable anions include chloride, bromide, iodide, sulfate, nitrate, phosphate, citrate, methanesulfonate, trifluoroacetate, acetate, malate, tosylate, tartrate, fumurate, glutamate, glucuronate, lactate, glutarate, and maleate. Likewise, a salt can also be formed between a cation and a negatively charged group on the compound, polymer, or agent. Suitable cations include sodium ion, potassium ion, magnesium ion, calcium ion, and an ammonium cation (e.g., tetramethylammonium ion). A precursor can be ester and another suitable derivative, which, during the process of preparing a polyurea or polyurethane capsule composition of this invention, is capable of converting to the compound, polymer, or agent and being used in preparing the polyurea or polyurethane capsule composition. A hydrate refers to the compound, polymer, or agent that contains water. A solvate refers to a complex formed between the compound, polymer, or agent and a suitable solvent. A suitable solvent can be water, ethanol, isopropanol, ethyl acetate, acetic acid, and ethanolamine.

Certain compounds, polymers, and agents have one or more stereocenters, each of which can be in the R configuration, the S configuration, or a mixture. Further, some compounds, polymers, and agents possess one or more double bonds wherein each double bond exists in the E (trans) or Z (cis) configuration, or combinations thereof. The compounds, polymers, and agents include all possible configurational stereoisomeric, regioisomeric, diastereomeric, enantiomeric, and epimeric forms as well as any mixtures thereof. As such, lysine used herein includes L-lysine, D-lysine, L-lysine monohydrochloride, D-lysine monohydrochloride, lysine carbonate, and so on. Similarly, arginine includes L-arginine, D-arginine, L-arginine monohydrochloride, D-arginine monohydrochloride, arginine carbonate, arginine monohydrate, and etc. Guanidine includes guanidine hydrochloride, guanidine carbonate, guanidine thiocyanate, and other guanidine salts including their hydrates. Ornithine include L-ornithine and its salts/hydrates (e.g., monohydrochloride) and D-ornithine and its salts/hydrates (e.g., monohydrochloride).

### Applications.

The capsules and the compositions of the present invention are well-suited for use, without limitation, in the following products:
a) Household products
   i. Liquid or Powder Laundry Detergents which can use the present invention include those systems described in U.S. Pat. Nos. 5,929,022, 5,916,862, 5,731,278, 5,565,145, 5,470,507, 5,466,802, 5,460,752, 5,458,810, 5,458,809, 5,288,431, 5,194,639, 4,968,451, 4,597,898, 4,561,998, 4,550,862, 4,537,707, 4,537,706, 4,515,705, 4,446,042, and 4,318,818
   ii. Unit Dose Pouches, Tablets and Capsules such as those described in EP 1 431 382 A1, US 2013/0219996 A1, US 2013/0284637 A1, and U.S. Pat. No. 6,492,315. These unit dose formulations can contain high concentrations of a functional material (e.g., 5-100% fabric softening agent or detergent active), fragrance (e.g., 0.5-100%, 0.5-40%, and 0.5-15%), and flavor (e.g., 0.1-100%, 0.1-40%, and 1-20%). They can contain no water to limit the water content as low as less than 30% (e.g., less than 20%, less than 10%, and less than 5%).
   iii. Scent Boosters such as those described in U.S. Pat. No. 7,867,968, U.S. Pat. No. 7,871,976, U.S. Pat. No. 8,333,289, US 2007/0269651 A1, and US2014/0107010 A1.
   iv. Fabric Care Products such as Rinse Conditioners (containing 1 to 30 weight % of a fabric conditioning active), Fabric Liquid Conditioners (containing 1 to 30 weight % of a fabric conditioning active), Tumble Drier Sheets, Fabric Refreshers, Fabric Refresher Sprays, Ironing Liquids, and Fabric Softener Systems such as those described in U.S. Pat. Nos. 6,335,315, 5,674,832, 5,759,990, 5,877,145, 5,574,179, 5,562,849, 5,545,350, 5,545,340, 5,411,671, 5,403,499, 5,288,417, 4,767,547 and 4,424,134 Liquid fabric softeners/fresheners contains at least one fabric softening agent present, preferably at a concentration of 1 to 30% (e.g., 4 to 20%, 4 to 10%, and 8 to 15%). The ratio between the active material and the fabric softening agent can be 1:500 to 1:2 (e.g., 1:250 to 1:4 and 1:100 to 1:8). As an illustration, when the fabric softening agent is 5% by weight of the fabric softener, the active material is 0.01 to 2.5%, preferably 0.02 to 1.25% and more preferably 0.1 to 0.63%. As another example, when the fabric softening agent is 20% by weight of the fabric softener, the active material is 0.04 to 10%, preferably 0.08 to 5% and more preferably 0.4 to 2.5%. The active material is a fragrance, malodor counteractant or mixture thereof. The liquid fabric softener can have 0.15 to 15% of capsules (e.g., 0.5 to 10%, 0.7 to 5%, and 1 to 3%). When including capsules at these levels, the neat oil equivalent (NOE) in the softener is 0.05 to 5% (e.g., 0.15 to 3.2%, 0.25 to 2%, and 0.3 to 1%).
      Suitable fabric softening agents include cationic surfactants. Non-limiting examples are quaternary ammonium compounds such as alkylated quaternary ammonium compounds, ring or cyclic quaternary ammonium compounds, aromatic quaternary ammonium compounds, diquaternary ammonium compounds, alkoxylated quaternary ammonium compounds, amidoamine quaternary ammonium compounds, ester quaternary ammonium compounds, and mixtures thereof. Fabric softening compositions, and components thereof, are generally described in US 2004/0204337 and US 2003/0060390. Suitable softening agents include esterquats such as Rewoquat WE 18 commercially available from Evonik Industries and Stepantex SP-90 commercially available from Stepan Company.
   v. Liquid dish detergents such as those described in U.S. Pat. Nos. 6,069,122 and 5,990,065
   vi. Automatic Dish Detergents such as those described in U.S. Pat. Nos. 6,020,294, 6,017,871, 5,968,881, 5,962,386, 5,939,373, 5,914,307, 5,902,781, 5,705,464, 5,703,034, 5,703,030, 5,679,630, 5,597,936, 5,581,005, 5,559,261, 4,515,705, 5,169,552, and 4,714,562
   vii. All-purpose Cleaners including bucket dilutable cleaners and toilet cleaners
   viii. Bathroom Cleaners
   ix. Bath Tissue
   x. Rug Deodorizers
   xi. Candles
   xii. Room Deodorizers
   xiii. Floor Cleaners
   xiv. Disinfectants
   xv. Window Cleaners
   xvi. Garbage bags/trash can liners
   xvii. Air Fresheners including room deodorizer and car deodorizer, scented candles, sprays, scented oil air freshener, Automatic spray air freshener, and neutralizing gel beads
   xviii. Moisture absorber
   xix. Household Devices such as paper towels and disposable Wipes
   xx. Moth balls/traps/cakes
b) Baby Care Products
   i. Diaper Rash Cream/Balm
   ii. Baby Powder
c) Baby Care Devices
   i. Diapers
   ii. Bibs
   iii. Wipes
d) Oral Care Products. Tooth care products (as an example of preparations according to the invention used for oral care) generally include an abrasive system (abrasive or polishing agent), for example silicic acids, calcium carbonates, calcium phosphates, aluminum oxides and/or hydroxylapatites, surface-active substances, for example sodium lauryl sulfate, sodium lauryl sarcosinate and/or cocamidopropylbetaine, humectants, for example glycerol and/or sorbitol, thickening agents, for example carboxymethyl cellulose, polyethylene glycols, carrageenan and/or Laponite®, sweeteners, for example saccharin, taste correctors for unpleasant taste sensations, taste correctors for further, normally not unpleasant taste sensations, taste-modulating substances (for example inositol phosphate, nucleotides such as guanosine monophosphate, adenosine monophosphate or other substances such as sodium glutamate or 2-phenoxypropionic acid), cooling active ingredients, for example menthol derivatives, (for example L-menthyllactate, L-menthylalkylcarbonates, menthone ketals, menthane carboxylic acid amides), 2,2,2-trialkylacetic acid amides (for example 2,2-diisopropylpropionic acid methyl amide), icilin and icilin derivatives, stabilizers and active ingredients, for example sodium fluoride, sodium monofluorophosphate, tin difluoride, quaternary ammonium fluorides, zinc citrate, zinc sulfate, tin pyrophosphate, tin dichloride, mixtures of various pyrophosphates, triclosan, cetylpyridinium chloride, aluminum lactate, potassium citrate, potassium nitrate, potassium chloride, strontium chloride, hydrogen peroxide, flavorings and/or sodium bicarbonate or taste correctors.
   i. Tooth Paste. An exemplary formulation as follows:
      1. calcium phosphate 40-55%
      2. carboxymethyl cellulose 0.8-1.2%
      3. sodium lauryl sulfate 1.5-2.5%
      4. glycerol 20-30%
      5. saccharin 0.1-0.3%
      6. flavor oil 1-2.5%
      7. water q.s. to 100%
         A typical procedure for preparing the formulation includes the steps of
         (i) mixing by a blender according to the foregoing formulation to provide a toothpaste, and (ii) adding a composition of this invention and blending the resultant mixture till homogeneous.
   ii. Tooth Powder
   iii. Oral Rinse
   iv. Tooth Whiteners
   v. Denture Adhesive
e) Health Care Devices
   i. Dental Floss
   ii. Toothbrushes
   iii. Respirators
   iv. Scented/flavored condoms
f) Feminine Hygiene Products such as Tampons, Feminine Napkins and Wipes, and Pantiliners
g) Personal Care Products: Cosmetic or pharmaceutical preparations, e.g., a “water-in-oil” (W/O) type emulsion, an “oil-in-water” (O/W) type emulsion or as multiple emulsions, for example of the water-in-oil-in-water (W/O/W) type, as a PIT emulsion, a Pickering emulsion, a micro-emulsion or nano-emulsion; and emulsions which are particularly preferred are of the “oil-in-water” (O/W) type or water-in-oil-in-water (W/O/W) type. More specifically,
   i. Personal Cleansers (bar soaps, body washes, and shower gels)
   ii. In-shower conditioner
   iii. Sunscreen ant tattoo color protection (sprays, lotions, and sticks)
   iv. Insect repellants
   v. Hand Sanitizer
   vi. Antiinflammatory balms, ointments, and sprays
   vii. Antibacterial ointments and creams
   viii. Sensates
   ix. Deodorants and Antiperspirants including aerosol and pump spray antiperspirant, stick antiperspirant, roll-on antiperspirant, emulsion spray antiperspirant, clear emulsion stick antiperspirant, soft solid antiperspirant, emulsion roll-on antiperspirant, clear emulsion stick antiperspirant, opaque emulsion stick antiperspirant, clear gel antiperspirant, clear stick deodorant, gel deodorant, spray deodorant, roll-on, and cream deordorant.
   x. Wax-based Deodorant. An exemplary formulation as follows:
      1. Parafin Wax 10-20%
      2. Hydrocarbon Wax 5-10%
      3. White Petrolatum 10-15%
      4. Acetylated Lanolin Alcohol 2-4%
      5. Diisopropyl Adipate 4-8%
      6. Mineral Oil 40-60%
      7. Preservative (as needed)
         The formulation is prepared by (i) mixing the above ingredients, (ii) heating the resultant composition to 75° C. until melted, (iii) with stirring, adding 4% cryogenically ground polymer containing a fragrance while maintaining the temperature 75° C., and (iv) stirring the resulting mixture in order to ensure a uniform suspension while a composition of this invention is added to the formulation.
   xi. Glycol/Soap Type Deodorant. An exemplary formulation as follows:
      1. Propylene Glycol 60-70%
      2. Sodium Stearate 5-10%
      3. Distilled Water 20-30%
      4. 2,4,4-Trichloro-2′-Hydroxy Diphenyl Ether, manufactured by the Ciba-Geigy Chemical Company and a Trademark of the Ciba-Geigy Chemical Company) 0.01-0.5%
         The ingredients are combined and heated to 75° C. with stirring until the sodium stearate has dissolved. The resultant mixture is cooled to 40° C. followed by addition of a composition of this invention.
   xii. Lotion including body lotion, facial lotion, and hand lotion
   xiii. Body powder and foot powder
   xiv. Toiletries
   xv. Body Spray
   xvi. Shave cream and male grooming products
   xvii. Bath Soak
   xviii. Exfoliating Scrub
h) Personal Care Devices
   i. Facial Tissues
   ii. Cleansing wipes
i) Hair Care Products
   i. Shampoos (liquid and dry powder)
   ii. Hair Conditioners (Rinse-out conditioners, leave-in conditioners, and cleansing conditioners)
   iii. Hair Rinses
   iv. Hair Refreshers
   v. Hair perfumes
   vi. Hair straightening products
   vii. Hair styling products, Hair Fixative and styling aids
   viii. Hair combing creams
   ix. Hair wax
   x. Hair foam, hair gel, nonaerosol pump spray
   xi. Hair Bleaches, Dyes and Colorants
   xii. Perming agents
   xiii. Hair wipes
j) Beauty Care
   i. Fine Fragrance—Alcoholic. Compositions and methods for incorporating fragrance capsules into alcoholic fine fragrances are described in U.S. Pat. No. 4,428,869. Alcoholic fine fragrances may contain the following:
      1. Ethanol (1-99%)
      2. Water (0-99%)
      3. A suspending aide including but not limited to: hydroxypropyl cellulose, ethyl cellulose, silica, microcrystalline cellulose, carrageenan, propylene glycol alginate, methyl cellulose, sodium carboxymethyl cellulose or xanthan gum (0-1%)
      4. Optionally an emulsifier or an emollient may be included including but not limited to those listed above
   ii. Solid Perfume
   iii. Lipstick/lip balm
   iv. Make-up cleanser
   v. Skin care cosmetic such as foundation, pack, sunscreen, skin lotion, milky lotion, skin cream, emollients, skin whitening
   vi. Make-up cosmetic including manicure, mascara, eyeliner, eye shadow, liquid foundation, powder foundation, lipstick and cheek rouge
k) Consumer goods packaging such as fragranced cartons, fragranced plastic bottles/boxes
l) Pet care products
   i. Cat litter
   ii. Flea and tick treatment products
   iii. Pet grooming products
   iv. Pet shampoos
   v. Pet toys, treats, and chewables
   vi. Pet training pads
   vii. Pet carriers and crates
m) Confectionaries confectionery, preferably selected from the group consisting of chocolate, chocolate bar products, other products in bar form, fruit gums, hard and soft caramels and chewing gum
   i. Gum
      1. Gum base (natural latex chicle gum, most current chewing gum bases also presently include elastomers, such as polyvinylacetate (PVA), polyethylene, (low or medium molecular weight) polyisobutene (PIB), polybutadiene, isobutene-isoprene copolymers (butyl rubber), polyvinylethylether (PVE), polyvinylbutyether, copolymers of vinyl esters and vinyl ethers, styrene-butadiene copolymers (styrene-butadiene rubber, SBR), or vinyl elastomers, for example based on vinylacetate/vinyllaurate, vinylacetate/vinylstearate or ethylene/vinylacetate, as well as mixtures of the mentioned elastomers, as described for example in EP 0 242 325, U.S. Pat. No. 4,518,615, U.S. Pat. No. 5,093,136, U.S. Pat. No. 5,266,336, U.S. Pat. No. 5,601,858 or U.S. Pat. No. 6,986,709.) 20-25%
      2. Powdered sugar 45-50%
      3. glucose 15-17%
      4. starch syrup 10-13%
      5. plasticizer 0.1%
      6. flavor 0.8-1.2%
         The components described above were kneaded by a kneader according to the foregoing formulation to provide a chewing gum. Encapsulated Flavor or sensate is then added and blended till homogeneous.
   ii. Breath Fresheners
   iii. Orally Dissolvable Strips
   iv. Chewable Candy
   v. Hard Candy
n) Baked products, preferably selected from the group consisting of bread, dry biscuits, cakes and other cookies;
o) snack foods, preferably selected from the group consisting of baked or fried potato chips or potato dough products, bread dough products and corn or peanut-based extrudates;
   i. Potato, tortilla, vegetable or multigrain chips
   ii. Popcorn
   iii. Pretzels
   iv. Extruded stacks
p) Cereal Products preferably selected from the group consisting of breakfast cereals, muesli bars and precooked finished rice products
q) Alcoholic and non-alcoholic beverages, preferably selected from the group consisting of coffee, tea, wine, beverages containing wine, beer, beverages containing beer, liqueurs, schnapps, brandies, sodas containing fruit, isotonic beverages, soft drinks, nectars, fruit and vegetable juices and fruit or vegetable preparations; instant beverages, preferably selected from the group consisting of instant cocoa beverages, instant tea beverages and instant coffee beverages
   i. Ready to drink liquid drinks
   ii. Liquid Drink Concentrates
   iii. Powder Drinks
   iv. Coffee: Instant Cappucino
      1. Sugar 30-40%
      2. Milk Powder 24-35%
      3. Soluble Coffee 20-25%
      4. Lactose 1-15%
      5. Food Grade Emulsifier 1-3%
      6. Encapsulated Volatile Flavor 0.01-0.5%
   v. Tea
   vi. Alcoholic
r) Spice blends and consumer prepared foods
   i. Powder gravy, sauce mixes
   ii. Condiments
   iii. Fermented Products
s) Ready to heat foods: ready meals and soups, preferably selected from the group consisting of powdered soups, instant soups, precooked soups
   i. Soups
   ii. Sauces
   iii. Stews
   iv. Frozen entrees
t) Dairy Products milk products, preferably selected from the group consisting of milk beverages, ice milk, yogurt, kefir, cream cheese, soft cheese, hard cheese, powdered milk, whey, butter, buttermilk and partially or fully hydrolyzed milk protein-containing products Flavored milk beverages
   i. Yoghurt
   ii. Ice cream
   iii. Bean Curd
   iv. Cheese
u) Soya protein or other soybean fractions, preferably selected from the group consisting of soya milk and products produced therefrom, soya lecithin-containing preparations, fermented products such as tofu or tempeh or products produced therefrom and soy sauces;
v) Meat products, preferably selected from the group consisting of ham, fresh or raw sausage preparations, and seasoned or marinated fresh or salt meat products
w) Eggs or egg products, preferably selected from the group consisting of dried egg, egg white and egg yolk
x) Oil-based products or emulsions thereof, preferably selected from the group consisting of mayonnaise, remoulade, dressings and seasoning preparations
y) fruit preparations, preferably selected from the group consisting of jams, sorbets, fruit sauces and fruit fillings; vegetable preparations, preferably selected from the group consisting of ketchup, sauces, dried vegetables, deep-frozen vegetables, precooked vegetables, vegetables in vinegar and preserved vegetables
z) Flavored pet foods.

The above-listed applications are all well known in the art. For example, fabric softener systems are described in U.S. Pat. Nos. 6,335,315, 5,674,832, 5,759,990, 5,877,145, 5,574,179; 5,562,849, 5,545,350, 5,545,340, 5,411,671, 5,403,499, 5,288,417, and 4,767,547, 4,424,134. Liquid laundry detergents include those systems described in U.S. Pat. Nos. 5,929,022, 5,916,862, 5,731,278, 5,565,145, 5,470,507, 5,466,802, 5,460,752, 5,458,810, 5,458,809, 5,288,431, 5,194,639, 4,968,451, 4,597,898, 4,561,998, 4,550,862, 4,537,707, 4,537,706, 4,515,705, 4,446,042, and 4,318,818. Liquid dish detergents are described in U.S. Pat. Nos. 6,069,122 and 5,990,065. Shampoo and conditioners that can employ the present invention include those described in U.S. Pat. Nos. 6,162,423, 5,968,286, 5,935,561, 5,932,203, 5,837,661, 5,776,443, 5,756,436, 5,661,118, 5,618,523, 5,275,755, 5,085,857, 4,673,568, 4,387,090 and 4,705,681. Automatic Dish Detergents are described in U.S. Pat. Nos. 6,020,294, 6,017,871, 5,968,881, 5,962,386, 5,939,373, 5,914,307, 5,902,781, 5,705,464, 5,703,034, 5,703,030, 5,679,630, 5,597,936, 5,581,005, 5,559,261, 4,515,705, 5,169,552, and 4,714,562.

As used herein olfactory effective amount is understood to mean the amount of compound in the capsule composition the individual components will contribute to its particular olfactory characteristics, but the olfactory effect of the capsule composition will be the sum of the effects of each of the individual components. Thus, the capsules of this invention can be used to alter the aroma characteristics of a consumer product, e.g., a fine perfume, by modifying the olfactory reaction contributed by another ingredient in the consumer product. The amount will vary depending on many factors including other ingredients, their relative amounts and the effect that is desired.

All parts, percentages and proportions refer to herein and in the claims are by weight unless otherwise indicated.

The values and dimensions disclosed herein are not to be understood as being strictly limited to the exact numerical values recited. Instead, unless otherwise specified, each such value is intended to mean both the recited value and a functionally equivalent range surrounding that value. For example, a value disclosed as “50%” is intended to mean “about 50%.”

The terms “capsule” and “microcapsule” herein are used interchangeably.

The terms “polyfunctional isocyanate,” “multifunctional isocyanate,” and “polyisocyanate” all refer to a compound having two or more isocyanate (—NCO) groups.

The terms “polyfunctional amine,” “multifunctional amine,” and “polyamine” refers to a compound containing two or more primary or secondary amine groups. These terms also refers to a compound containing one or more primary/secondary amine groups and one or more hydroxyl groups (—OH).

The terms “polyfunctional alcohol,” “multifunctional alcohol,” “poly alcohol,” and “polyol” refer to a compound having two or more hydroxyl groups.

The invention is described in greater detail by the following non-limiting examples. Without further elaboration, it is believed that one skilled in the art can, based on the description herein, utilize the present invention to its fullest extent. All publications cited herein are incorporated by reference in their entirety.

### Examples 1-12

Fifteen capsule compositions of this invention, i.e., Compositions 1-12, were prepared in these examples, following the procedure below.

More specifically, fragrance Greenfields (192 g; International Flavors and Fragrance Inc., Union Beach, N.J.) was mixed with NEOBEE oil (48 g; commercially available from Stepan Company, Northfield, Ill.) and Lupranate M20 (19.2 g; polymeric methylene diphenyl diisocyanate; commercially available from BASF, Mannheim, Germany), to form an oil phase. Subsequently, the oil phase was emulsified into 319.2 g of an aqueous dispersant solution containing 0.94% Mowiol 4-98 (a PVA having a degree of hydrolysis of 98-99 mol % and a molecular weight of 27,000 Daltons; commercially available from Kuraray, Chiyoda-Ku, Japan) and 0.94% Walocel CRT 50000 PA (CMC commercially available from Dow Chemical Company, Midland, Mich.) under high shearing (IKA—ULTRA TURRAX, T25 Basic) at 9500 rpm for three minutes. The resultant fragrance emulsion was heated to 35° C. and 21.6 g of 40% hexamethylene diamine aqueous solution was added under constant mixing using an overhead mixer. After 15 minutes at 35° C., a capsule slurry was formed and consequently cured at 55° C. for two hours and then cooled to room temperature to obtain capsule Composition 1.

In Example 2, Composition 2 was prepared following the same procedure as described in Example 1, except that the dispersant solution contained 0.4% (instead of 0.94%) Mowiol 4-98 and 0.94% Walocel CRT 50000 PA.

In Example 3, Composition 3 was prepared following the same procedure as descried in Example 1, except that the dispersant solution contained 0.6% (instead of 0.94%) Mowiol 4-98 and 0.94% Walocel CRT 50000 PA.

In Example 4, Composition 4 was prepared following the same procedure as descried in Example 1, except that the dispersant solution contained 0.8% (instead of 0.94%) Mowiol 4-98 and 0.94% Walocel CRT 50000 PA.

In Example 5, Composition 5 was prepared following the same procedure as descried in Example 1, except the dispersant solution contained 0.94% Mowiol 4-98 and 0.8% (instead of 0.94%) Walocel CRT 50000 PA.

In Example 6, Composition 6 was prepared following the same procedure as descried in Example 1, except that the dispersant solution contained 0.94% Mowiol 4-98 and 0.6% (instead of 0.94%) Walocel CRT 50000 PA.

In Example 7, Composition 7 was prepared following the same procedure as descried in Example 1, except that the dispersant solution contained 0.94% Mowiol 4-98 and 0.4% (instead of 0.94%) Walocel CRT 50000 PA.

In Example 8, Composition 8 was prepared following the same procedure as descried in Example 1, except that the dispersant solution contained 0.94% Mowiol 4-98 and 0.2% (instead of 0.94%) Walocel CRT 50000 PA.

In Example 9, Composition 9 was prepared following the same procedure as descried in Example 1, except that (i) Fragrance Jillz, instead of Greenfields, was used and (ii) the dispersant solution contained 0.8% (instead of 0.94%) Mowiol 4-98 and 0.8% Glycoid 3S (cold-water-soluble purified tamarind seed gum; commercially available DSP Gokyo) instead of Walocel CRT 50000 PA.

In Example 10, Composition 10 was prepared following the same procedure as descried in Example 9, except that the dispersant solution contained 0.94% Mowiol 4-98 and 0.94% Merquat 3940 (polyquaternium-39; ampholytic terpolymer of acrylic acid, acrylamide and diallyldimethylammonium chloride; commercially available from Lubrizol) instead of Glycoid 3S.

In Example 11, Composition 11 was prepared following the same procedure as descried in Example 1, except that the dispersant solution contained 0.8% Mowiol 4-98, 0.8% Walocel CRT 50000 PA, and 0.2% Glycoid 3S.

In Example 12, Composition 12 was prepared following the same procedure as descried in Example 1, except that the dispersant solution contained 0.8% Mowiol 4-98, 0.8% Walocel CRT 50000 PA, and 0.2% alginic acid (Protonal commercially available from FMC BioPolymer), instead of 0.9% Mowiol 4-98 and 0.8% Walocel CRT 50000 PA.

Seven comparative compositions were also prepared to compare their performance with that of compositions of this invention.

Comparative 1 was prepared following the same procedure as in Example 1 except that the dispersant solution contained 0.94% Mowiol 3-85 (partially hydrolyzed PVA having a degree of hydrolysis of 85%; commercially available from Kuraray) and 0.94% Walocel CRT 50000 PA.

Comparative 2 was prepared following the same procedure as in Example 1 except that the dispersant solution contained 0.94% Mowiol 3-85.

Comparative 3 was prepared by mixing 20 g of Comparative 2 with an aqueous solution containing 0.94% Mowiol 4-98 and 0.94% Walocel CRT 50000 PA. The mixture was stirred for 30 minutes to obtain Comparative 3.

Comparative 4 was prepared following the same procedure as in Example 1 except that the dispersant solution contained 0.94% Walocel CRT 50000 PA without any Mowiol 4-98.

Comparative 5 was prepared following the same procedure as in Example 1 except that the dispersant solution contained 0.94% Mowiol 4-98 without any Walocel CRT 50000 PA.

### Example 13

Composition 13 was prepared following the same procedure as described in Example 1, except that the dispersant solution contained 1.4% Mowiol 4-98 and 0.9% Walocel CRT 50000 PA.

192 g of a fragrance Greenfields was weighed out and combined with 48 g of NEOBEE oil and 19.2 g of isocyanate, Lupranate M20 (BASF), to form an oil phase. In a separate beaker, an aqueous solution of 319.2 g of 1.4% Mowiol 4-98 (Kuraray) and 0.9% Walocel CRT 50000 PA (Dow) was prepared and then emulsified with the oil phase to form a fragrance emulsion under high shearing (IKA—ULTRA TURRAX, T25 Basic) at 9500 rpm for three minutes. The fragrance emulsion was heated to a 35° C. and 21.6 g of 40% hexamethylene diamine (commercially available from Sigma-Aldrich, St. Louis, Mo.) was added under constant mixing with an overhead mixer. After 15 minutes of stirring at 35° C., the capsule slurry was cured at 55° C. for two hours and then cooled to room temperature to obtain Composition 13.

### Example 14

Composition 14 was prepared following the same procedure as described in Example 1, except that the dispersant solution contained 0.9% Mowiol 4-98 and 0.9% Walocel CRT 50000 PA and 0.9% hydroxypropyl cellulose (commercially available from TCI America).

192 g of a fragrance Greenfields was weighed out and combined with 48 g of NEOBEE oil (Stepan) and 9.6 g of isocyanate, Lupranate M20 (BASF), to form an oil phase. In a separate beaker, an aqueous solution of 319.2 g of 0.9% Mowiol 4-98 (Kuraray), 0.9% Walocel CRT 50000 PA (Dow) and 0.9% hydroxypropyl cellulose (TCI America) was prepared and then emulsified with oil phase to form a fragrance emulsion under high shearing (IKA—ULTRA TURRAX, T25 Basic) at 9500 rpm for three minutes. The fragrance emulsion was then heated to a 35° C. Consequently, 10.8 g of 40% hexamethylene diamine (Sigma-Aldrich) and 20.4 g of water was added under constant mixing with an overhead mixer. After 15 minutes of stirring at 35° C., the capsule slurry was cured at 55° C. for two hours and then cooled to room temperature to obtain Composition 14.

### Example 15

Composition 15 was prepared following the same procedure as described in Example 1, except that the dispersant solution contained 0.9% Mowiol 4-98 and 0.9% hydroxypropyl cellulose.

180 g of a fragrance Greenfields was weighed out and combined with 45 g of NEOBEE oil (Stepan) and 9.6 g of isocyanate, Lupranate M20 (BASF), to form the oil phase. In a separate beaker, an aqueous solution of 200 g of 0.9% Mowiol 4-98 (Kuraray) and 0.9% Walocel CRT 50000 PA (Dow) was prepared and then emulsified with the previously prepared oil phase to form the fragrance emulsion under high shearing (IKA—ULTRA TURRAX, T25 Basic) at 9500 rpm for two minutes. To the fragrance emulsion was then added 60 g of 5.0% hydroxypropyl cellulose under constant mixing. The mixture was then heated to a 35° C. Next 10.8 g of 40% hexamethylene diamine (Sigma-Aldrich) and 94.6 g of distilled water was added under constant mixing with an overhead mixer. After 15 minutes of stirring at 35° C., the capsule slurry was cured at 55° C. for two hours and then cooled to room temperature to obtain Composition 15.

### Example 16

Composition 16 was prepared following the same procedure as described in Example 14, except that no CMC was used.

180 g of a fragrance Greenfields (International Flavors and Fragrance Inc., Union Beach) was weighed out and combined with 45 g of NEOBEE oil (Stepan) and 19.2 g of isocyanate, Lupranate M20 (BASF), to form the oil phase. In a separate beaker, an aqueous solution of 200 g of 2.4% Mowiol 4-98 (Kuraray) and 1.5% hydroxylpropyl cellulose was prepared and then emulsified with the previously prepared oil phase to form the fragrance emulsion under high shearing (IKA—ULTRA TURRAX, T25 Basic) at 9500 rpm for two minutes. The fragrance emulsion was then heated to a 35° C. Next 21.6 g of 40% hexamethylene diamine (Sigma-Aldrich) and 24.2 g of water was added under constant mixing with an overhead mixer. After 15 minutes of stirring at 35° C., the capsule slurry was cured at 55° C. for two hours and then cooled to room temperature to obtain Composition 16.

### Example 17

Composition 17 was prepared following the same procedure as described in Example 13 except that 50.4 g of 30% branched polyethylenimine was used instead of hexamethylene diamine.

### Example 18

Composition 17 was prepared following the same procedure as described in Example 13 except that different polyisocyanate and cross-linking agent were use.

180 g of a fragrance Greenfields was weighed out and combined with 45 g of NEOBEE oil (Stepan) and 19.2 g of aliphatic polyisocyanate prepolymer (Takenate D110-N), to form the oil phase. In a separate beaker, an aqueous solution of 200 g of 2.4% Mowiol 4-98 (Kuraray) and 1.5% Walocel CRT 50000 PA (Dow) was prepared and then emulsified with the previously prepared oil phase to form the fragrance emulsion under high shearing (IKA—ULTRA TURRAX, T25 Basic) at 9500 rpm for three minutes. The fragrance emulsion was heated to a 35° C. and 50.4 g of 30% branched polyethylenimine and 25.4 g of water was added under constant mixing with an overhead mixer. After 15 minutes of stirring at 35° C., the capsule slurry was cured at 55° C. for two hours and then cooled to room temperature to obtain Composition 18.

### Example 19

A spray-dried composition, Composition 19, was prepared following the procedure below.

1 kg of detergent powder for machine wash (commercially available from Persil Bio) or Hand Wash (Commercially available from BioTex Green Handwash, Houston, Tex.) was subjected to a rotating drum LD-1528 1 kg from Lothar A. Wolf Specialmaschinen GmBH (Germany) until homogenous powder was obtained. 6.7 g of Composition 1 was then sprayed onto the powder in the rotating drum by using a spray paint gun (Anest Iwata W-300), operated at 1-2 bar, to obtain Composition 19.

### Example 20

Another spray-dried composition, Composition 20, was prepared following the procedure below.

1 kg of detergent powder for Machine Wash (Persil Bio) or Hand Wash (BioTex Green Handwash) was subjected to a rotating drum LD-1528 1 kg from Lothar A. Wolf Specialmaschinen GmBH until homogenous powder was obtained. 6 g of Composition 1 mixed with 0.7 g of water was then sprayed onto the powder in the rotating drum by using a spray paint gun (Anest Iwata W-300), operated at 1-2 bar, to obtain Composition 20.

### Example 21

1 kg of detergent powder for Machine Wash (Persil Bio) or Hand Wash (BioTex Green Handwash) was subjected to a rotating drum LD-1528 1 kg from Lothar A. Wolf Specialmaschinen GmBH until homogenous powder was obtained. 6.7 g of Composition 1 mixed with 0.7 g of calcium chloride was then sprayed onto the powder in the rotating drum by using a spray paint gun (Anest Iwata W-300), operated at 1-2 bar, to obtain Composition 21.

### Examples 22-25

Four capsule aggregates, Compositions 22-25, were prepared following the procedure below.

Composition 12, containing alginate as the water soluble polymer, was used to prepare the capsule aggregate by adding multivalent cations to the capsule slurry.

Ca²⁺ was used as the multivalent cation to prepare Compositions 22 and 23. Composition 12 was first diluted to 10% with water. To the resultant mixture at stirring, was then added calcium chloride dihydrate (10% aqueous solution) to the level of 0.1% by weight of the composition. The aggregation formation was monitored by optical microscope. Composition 22 was obtained after the addition of CaCl₂ was complete. Composition 23 was prepared following the same preocedure as Composition 22 except that 0.2% (instead of 0.1%) of CaCl₂ was added.

Al³⁺ was used as the multivalent cation to prepare Compositions 24 and 25. The above procedure was followed except that aluminum sulfate hydate, instead of calcium chloride dehydrate, was added to a level of 0.1% for Composition 24, or 0.2% for Composition 25. Optical microscope confirmed the formation of aggregates.

### Microscopy Image

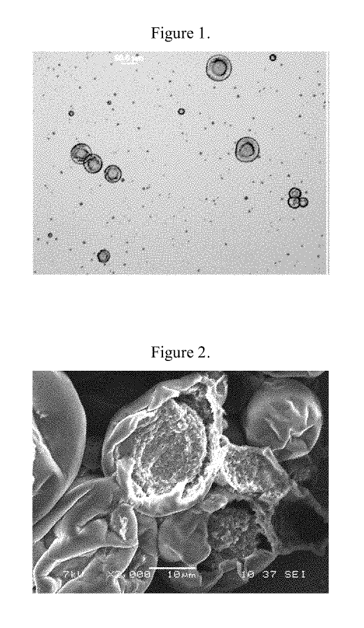

The microscopy image of Composition 1 was taken using an Olympus microscope, Model BX51. See FIG. 1. Before taking the image, a drop of 1% diluted sample of Composition 1 was placed on microscope slide with glass cover slip. The cover slip was compressed lightly with a metal spatula.

As shown in FIG. 1, the capsule walls in this figure each have an outer layer and an inner layer.

### Scanning Electron Microscopy Image (SEM)

The image (FIG. 2) was taken and shows an outer layer and an inner layer.

### Zeta Potential Measurement

To evaluate the bonding of the dispersant to the capsules, zeta potentials were measured for Composition 1 of this invention and also for a comparative composition, i.e., Comparative 3.

More specifically, 8 g of freshly prepared Composition 1 was added to water (4 g), mixed, and then centrifuged at 3400 rpm for 30 minutes. The aqueous layer was removed and another 4 g of water was added to Composition 1. The resultant wash sample, i.e., 1× Wash, was measured on a Malvern Zetasizer. The results are shown in Table 2 below. After the measurement, the 1× Wash was centrifuged again at 3400 rpm for 30 minutes. The aqueous layer was removed and 4 g of water was again added to Composition 1 to obtain a second wash sample, i.e., 2× Wash. The zeta potential was measured. See below Table 2.

TABLE 2

| | Zeta Potential (mV) | | |
|---|---|---|---|
| Sample | Initial | 1x Wash | 2x Wash |
| Composition 1 | −32.7 | −34.8 | −34.6 |
| Comparative 3 | −33.9 | −17.9 | −14.9 |

Comparative 3 was subjected to the same wash cycles and its zeta potentials were measured and compared to those of Composition 1.

As shown above, Composition 1 was prepared using two polymers, Mowiol 4-98 and Walocel CRT 50000 PA, as the dispersant. These two polymers bond to the capsules tightly so that they were not removed by washing the capsule slurry with water. The tight bonding was confirmed by the fact that the zeta potentials of Composition 1 changed less than 10% before and after being washed with water. The small changes are the result of changes in ionic strength, pH value, salt concentration, and etc. By contrast, to obtain Comparative 3, the same amounts of the two polymers were added to a capsule slurry, instead of using them as the dispersant to prepare the capsule slurry. Washing Comparative 3 with water removed the two polymers from the capsule slurry, indicating that Mowiol 4-98 and Walocel CRT 50000 PA do not tightly bond to the capsules. Their removal from the capsules by water resulted in the significant zeta potential changes (a decrease of more than 40%). obtained the same amount of Mowiol 4-98 and Walocel CRT 50000 PA.

The zeta potential results clearly showed that the fully hydrolyzed PVA and the water-dispersible polymer CMC are immobilized on the capsule wall to the extent that washing the capsule with water is incapable of removing them from the capsule.

### Particle Size Measurement

Particle sizes of the capsules in Compositions 1-8 and Comparatives 1, 2, 4, and 5 were measured following a standard procedure. See, e.g., WO 2009/153695 A1. To measure the particle sizes, 2 g of a composition was mixed with 100 g of water, which was subject to a measurement using a Malvern Mastersizer. The results were summarized in Table 3 below.

TABLE 3

| Composition | Ratio of PVA to CMC | Particle Size (μm) |
|---|---|---|
| 1 | 1:1* | 41 |
| 2 | 2:5* | 120 |
| 3 | 3:5* | 74 |
| 4 | 4:5* | 56 |
| 5 | 5:4* | 35 |
| 6 | 5:3* | 12 |
| 7 | 5:2* | 10 |
| 8 | 5:1* | 11 |
| Comparative 1 | 1:1*** | 7 |
| Comparative 2 | 5:0*** | 7 |
| Comparative 4 | 0:5* | NA** |
| Comparative 5 | 5:0* | NA** |

*Mowiol 4-98: Walocel CRT 50000 PA
**Could not make stable capsules
***Mowiol 3-85: Walocel CRT 50000 PA

Note that the particle sizes of the capsules in Compositions 9-18 were also measured and shown in Table 8 below.

As shown in Table 3 above, stable capsules could not be prepared by using either the fully hydrolyzed PVA (Mowiol 4-98) or the water-dispersible polymer CMC alone (see Comparatives 4 and 5 in the above table).

Further, the particle size can be predetermined by using a certain ratio of a fully hydrolyzed PVA to the water-dispersible polymer. See Table 3, Compositions 1-8. The capsule particle size is 11 microns when the ratio of a fully hydrolyzed PVA to CMC is 5:1 and raises to 120 microns when the ratio of the fully hydrolyzed PVA to CMC changes to 2:5. By contrast, when a partially hydrolyzed PVA is used instead of the fully hydrolyzed PVA, the capsule particle size remained the same when changing the ratio of the partially hydrolyzed PVA to CMC.

### Fragrance Intensity in a Liquid Detergent Base

The performance of Compositions 1 and 13-18 was evaluated in a liquid detergent base. More specifically, Composition 1 was blended into a model un-fragranced liquid detergent base at 0.5% fragrance oil equivalent and at 2500 rpm for 3 minutes.

The resulting base was applied to a standard European washing machine protocol with towels as described in U.S. Pat. No. 8,299,011. The towels were line-dried for 24 hours followed by sensory evaluation by a panel of judges. The fragrance intensity was rated on a scale ranging from 0 to 10 pre- and post-rubbing the towel swatches. A numerical value of 5 indicated the towel producing a strong intensity, while a value of 10 indicated the towel generating a very strong smell.

The results are shown in Tables 4 and 5 below. In Table 4, Composition 1 of this invention had a post-rub fragrance intensity of 4.44. By contrast, Comparative Compositions 1, 2, and 3 had a post-rub fragrance intensity of 1.56, 1.78, and 2.39, respectively. Composition 1 had, unexpectedly, higher pre-rub and much higher post-rub fragrance intensities than each of the three comparative compositions evaluated in this study. Table 5 shows the fragrance intensities for Compositions 13-18.

TABLE 4

Liquid Detergent
Performance (Intensity)

| Composition | Pre-rub | Post-rub |
|---|---|---|
| 1 | 0.63 | 4.44 |
| Comparative 1 | 0.17 | 1.56 |
| Comparative 2 | 0.28 | 1.78 |
| Comparative 3 | 0.22 | 2.39 |

TABLE 5

| | Liquid Detergent | | |
|---|---|---|---|
| | Performance (Intensity) | | |
| Composition | Damp | Pre-rub | Post-rub |
| 13 | 2.1 | 1.2 | 3.5 |
| 14 | 2.8 | 0.6 | 2.3 |
| 15 | 3.3 | 0.7 | 1.3 |
| 16 | 2.8 | 1.1 | 3.6 |
| 17 | 2.0 | 0.6 | 2.7 |
| 18 | 1.6 | 0.8 | 4.8 |

### Fragrance Intensity in Powder Detergent Base (Machine Wash)

The performance of solid Compositions 19, 20, 21 was evaluated using a standard European washing machine protocol with towels as described in U.S. Pat. No. 8,299,011. 100 g of Composition 19, 20, 21 at 0.2% fragrance oil equivalence were tested. The towels were first sensory evaluated by a panel of judges at damp when taken out of the machine. Next the towels were line-dried for 24 hours followed by sensory evaluations by a panel of judges. The fragrance intensity was rated on a scale ranging from 0 to 5 pre- and post-rubbing the towel swatches. A numerical value of 3 indicated the towel producing a strong intensity, while 5 indicated the towel generated a very strong smell.

Results are shown in Table 6 below. In the table, Composition 19 of the invention had fragrance intensities of 2.8, 1.5 and 2.6, at damp, dry pre-rub and post-rub, respectively.

TABLE 6

| | Fragrance Intensity | | |
|---|---|---|---|
| Composition | Damp | Pre-rub | Post-rub |
| 19 | 2.8 | 1.5 | 2.6 |
| 20 | 2.2 | 1.0 | 1.6 |
| 21 | 2.4 | 1.0 | 1.7 |

### Fragrance Intensity in Powder Detergent Base (Hand Wash)

The performance of Compositions 19, 20, 21 was further evaluated using a following hand washing protocol. To stainless-steel basin was added 2 liters of water at 20° C. 10 g of Composition 19, 20, 21 at 0.2% fragrance oil equivalence were then dissolved in the water. 4 towels were then added and soaked for 25-30 minutes. Towels were folded then put through the mangle twice. The towels were then rinsed twice with 2 liters of clean water. The towels were first sensory evaluated by a panel of judges at damp when taken out of the machine. Next the towels were line-dried for 24 hours followed by sensory evaluations by a panel of judges. The fragrance intensity was rated on a scale ranging from 0 to 5 pre- and post-rubbing the towel swatches. A numerical value of 3 indicated the towel producing a strong intensity, while 5 indicated the towel generated a very strong smell.

Results are shown in Table 7 below. In the table, Composition 19 of the invention had the highest fragrance intensity of 2.9 at post-rub. Whereas Composition 20 of the invention had consistent benefit at damp, dry pre- and post-rub.

TABLE 7

| | Fragrance Intensity | | |
|---|---|---|---|
| Composition | Damp | Pre-rub | Post-rub |
| 19 | 1.5 | 1.9 | 2.9 |
| 20 | 2.0 | 2.4 | 1.8 |
| 21 | 1.6 | 1.9 | 1.6 |

### Encapsulation Efficiency

Compositions 9-18 were evaluated for their encapsulation efficiency. Free oil in the capsule slurry was analyzed and its contents were used to calculate encapsulation efficiency summarized in Table 8 below. The encapsulation efficiency (%) equals to (% total fragrance oil−% free oil)×100/% total fragrance oil. The percentages here each refer to the percentage by the weight of the capsule composition.

All four compositions showed an unexpectedly high encapsulation efficiency. Composition 9 and 10 each had only 0.05% free oil in the slurry compared to the weight the total fragrance oil, Composition 11 had 0.1% free oil, and Composition 12 had 0.2% free oil.

TABLE 8

| | Encapsulation | Particle Size - |
|---|---|---|
| COMPOSITIONS | Efficiency (%) | Mode (μm) |
| 9 | 99.8 | 62 |
| 10 | 99.8 | 117 |
| 11 | 99.7 | 44 |
| 12 | 99.4 | 43 |
| 13 | 99.7 | 40 |
| 14 | 99.4 | 12 |
| 15 | 97.7 | 48 |
| 16 | 99.8 | 15 |
| 17 | 99.8 | 40 |
| 18 | 99.8 | 50 |

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

Indeed, to achieve the purpose of preparing a capsule composition containing a fully hydrolyzed PVA and a water-dispersible polymer, one skilled in the art can design and prepare a capsule composition by using different water-dispersible polymers, capsule-wall forming materials including different polyisocyanate, amine crosslinkers, alcohol crosslinkers, and other optional components. Further, the ratios among the wall-forming materials, the fully hydrolyzed PVA, and the water-dispersible polymers can also be determined by a skilled artisan through assays described in this application or those known in the art. The particle size of the capsules can be controlled by adjusting the ratio of the fully hydrolyzed PVA to the water-dispersible polymer, by varying the concentrations of the wall-forming materials, and by using a co-dispersant, which can be readily determined by a skilled artisan after certain optimization.

From the above description, a skilled artisan can easily ascertain the essential characteristics of the present invention, and without departing from the spirit and scope thereof, can make various changes and modifications of the invention to adapt it to various usages and conditions. Thus, other embodiments are also within the claims.

What is claimed is:

1. A method of preparing a capsule composition, the method comprising:
(a) providing an oil phase containing an active material and a wall-forming material;
(b) providing an aqueous phase containing a dispersant that includes a fully hydrolyzed polyvinyl alcohol and a water-dispersible polymer;
(c) emulsifying the oil phase into the aqueous phase to form an oil-in-water emulsion;
(d) optionally adding an activation agent to the oil-in-water emulsion;
(e) causing the formation of capsules having an oil core that contains the active material and a capsule wall that is formed of the wall-forming material; and
(f) curing the capsules to obtain the capsule composition,
wherein the weight ratio between the fully hydrolyzed polyvinyl alcohol and the water-dispersible polymer is 1:10 to 100:1, the capsules each have a particle size of 0.1 to 1000 microns, and the particle size is adjustable by varying the weight ratio between the fully hydrolyzed polyvinyl alcohol and the water-dispersible polymer.

2. The method of claim 1, wherein the fully hydrolyzed polyvinyl alcohol has a degree of hydrolysis of 96% or greater; and the water-dispersible polymer is a polysaccharide, protein, polypeptide, polyacrylate, polyolefin, polyurethane, polyurea, polyamide, polyalkylene oxide, polysiloxane, polyamine, or combination thereof.

3. The method of claim 2, wherein the polysaccharide is agar, carboxymethylcellulose, carboxyethylcellulose, alginic acid, xyloglucan, xanthum gum, gum Arabic, hydroxypropyl cellulose, hydroxyethyl cellulose, carrageenan, modified starch, modified cellulose, galactomannans, amphoteric guar, hydrophobically modified cationic guar, hydrophobically modified amphoteric guar, hydrophobically modified anionic guar, bacterial alginate, fucogalactan, fucoidan, gellan gum, gum ghatti, gum karaya, gum tragacanth, pectin, propylene glycol alginate, psyllium seed gum, sodium alginate, welan gum, or combination thereof.

4. The method of claim 1, wherein the capsules each have a particle size of 5 to 150 microns, the weight ratio between the fully hydrolyzed polyvinyl alcohol and the water soluble polymer is 1:5 to 10:1, and the capsule wall is formed of a polyacrylate, polyurea, polyurethane, polyacrylamide, polyester, polyether, polyamide, poly(acrylate-co-acrylamide), starch, silica, gelatin and gum Arabic, poly(melamine-formaldehyde), poly(urea-formaldehyde), or combination thereof.

5. The method of claim 4, wherein the wall-forming material is a polyisocyanate and the activation agent is an amine cross-linker, an alcohol cross-linker, or a hybrid cross-linker, the amine cross-linker containing two or more amine groups, the alcohol cross-linker containing two or more hydroxyl groups, and the hybrid cross-linker containing one or more amine groups and one or more hydroxyl groups; the capsules are cured at 45° C. to 130° C.; and the active material is a fragrance, flavor, malodor counteracting agent, or combination thereof.

6. The method of claim 1, wherein the capsule wall has an outer layer and an inner layer.

7. The method of claim 1, further comprising the step of (g): adding a salt of a multivalent cation after step (f) to form aggregates each containing two or more capsules, wherein the water-dispersible polymer contains alginic acid, an alginate salt, or a poly(methyl vinyl ether-co-maleic acid); the salt is a salt of calcium, magnesium, aluminum, iron, manganese, zinc, cobalt, copper, nickel, titanium, chromium, vanadium, gold, or a combination thereof; and the weight ratio between the salt and the water-dispersible polymer is 1:50,000 to 50,000:1.

8. The method of claim 1, further comprising the step of (h): spray drying the capsules.

9. A capsule composition prepared by a method of claim 1.

10. A capsule comprising:
an oil core having an active material;
an capsule wall formed of a wall-forming material, the capsule wall encapsulates the oil core; and
a dispersant containing a fully hydrolyzed polyvinyl alcohol and a water-dispersible polymer, both of which are immobilized on the capsule wall.

11. The capsule of claim 10, wherein the weight ratio between the fully hydrolyzed polyvinyl alcohol and the water-dispersible polymer is 1:10 to 100:1; the capsules each have a size of 0.1 to 1000 microns; the fully hydrolyzed polyvinyl alcohol has a degree of hydrolysis of 96% or greater; the water-dispersible polymer is a polysaccharide, protein, polypeptide, polyacrylate, polyolefin, polyurethane, polyurea, polyamide, polyalkylene oxide, polysiloxane, polyamine, or combination thereof; and the active material is a fragrance, flavor, malodor counteracting agent, or combination thereof.

12. The capsule of claim 11, wherein the polysaccharide is agar, carboxymethylcellulose, carboxyethylcellulose, alginic acid, xyloglucan, xanthum gum, gum Arabic, hydroxypropyl cellulose, hydroxyethyl cellulose, carrageenan, modified starch, modified cellulose, galactomannans, amphoteric guar, hydrophobically modified cationic guar, hydrophobically modified amphoteric guar, hydrophobically modified anionic guar, bacterial alginate, fucogalactan, fucoidan, gellan gum, gum ghatti, gum karaya, gum tragacanth, pectin, propylene glycol alginate, psyllium seed gum, sodium alginate, welan gum, or combination thereof.

13. The capsule of claim 10, wherein the capsule has a particle size of 5 to 150 microns and the weight ratio between the fully hydrolyzed polyvinyl alcohol and the water soluble polymer is 1:5 to 10:1.

14. The capsule of claim 10, wherein the capsule wall is formed of polyacrylate, polyurea, polyurethane, polyacrylamide, polyester, polyether, polyamide, poly(acrylate-co-acrylamide), starch, silica, gelatin and gum Arabic, poly(melamine-formaldehyde), poly(urea-formaldehyde), or combination thereof.

15. The capsule of claim 14, wherein the capsule wall is formed of polyurea, polyurethane, or combination thereof.

16. The capsule of claim 10, wherein the capsule wall has an outer layer and an inner layer.

17. A consumer product comprising a capsule of claim 10.

18. The consumer product of claim 17, further comprising one or more different capsules, a free active material, a deposition aid, or a combination thereof.

19. The consumer product of claim 17, wherein the consumer product is a shampoo, hair conditioner, personal wash, fabric detergent, softener, powder detergent, scent booster, bar soap, or hard surface cleaner.

20. The consumer product of claim 19, wherein the consumer product is a fabric detergent containing a fabric detergent active.
